(19) United States
(12) Patent Application Publication
(10) Pub. No.: US 2013/0174297 A1
(43) Pub. Date: Jul. 4, 2013

(54) CONTROLLING TGBp3 AND SILENCING bZIP60 TO REGULATE UPR

(71) Applicant: The Board of Regents for Oklahoma State Universi, Stillwater, OK (US)

(72) Inventors: JEANMARIE VERCHOT-LUBICZ, Stillwater, OK (US); CHANG-MING YE, Guangzhou (CN)

(73) Assignee: THE BOARD OF REGENTS FOR OKLAHOMA STATE UNIVERSITY, Stillwater, OK (US)

(21) Appl. No.: 13/655,260

(22) Filed: Oct. 18, 2012

Related U.S. Application Data
(60) Provisional application No. 61/548,625, filed on Oct. 18, 2011.

(51) Int. Cl. C12N 15/113

(52) U.S. Cl. CPC C12N 15/1131
USPC 800/286; 800/285; 800/301; 435/375

(57) ABSTRACT

Methods for treating, preventing or slowing viral spread between plants cells by suppressing expression of the bZIP60 gene and/or activity of the BZIP60 protein are provided. In another aspect, the present disclosure provides methods for delivering TGBp3 to cells to induce apoptosis, and/or to trigger pro-survival pathways.

### CROSS-REFERENCE TO RELATED APPLICATIONS

This application claims benefit of U.S. provisional patent application 61/548,625, filed Oct. 18, 2011, the complete contents of which are hereby incorporated by reference.

### SEQUENCE LISTING

This application includes as the Sequence Listing the complete contents of the accompanying text file “Sequence.txt”, created Oct. 18, 2012, containing 10,940 bytes, hereby incorporated by reference.

### FIELD

The present disclosure generally relates to the prevention and treatment of viral infections in plants. In particular, the present disclosure provides methods for treating, preventing or slowing viral spread between cells by suppressing expression of the bZIP60 gene and/or activity of the BZIP60 protein. Also provided are methods for providing TGBp3 to cells to induce apoptosis, and/or to trigger pro-survival pathways.

### BACKGROUND

Viral infections in plants are known to cause extensive damage. This is especially problematic in commercially valuable crops such as those which are grown for food, for animal feed, and for ornamentation. Single strand RNA (ssRNA) viruses are known to be the causative agent of many infections, and there is an ongoing need to provide methods for preventing, treating and containing the spread of ssRNA infections in plants.

### BRIEF SUMMARY

Provided herein are methods of treating, preventing or slowing viral infection in a plant. The methods comprise the step of inhibiting expression of a bZIP60 gene in said plant. In some embodiments, the step of inhibiting is performed by providing to said plant at least one agent that inhibits bZIP60 expression. The at least one agent that inhibits bZIP60 expression may be, for example: co-suppressive RNA, dsRNA, antisense RNA, hairpin RNA, intron containing hairpin RNA, an amplicon mediated interference agent, catalytic RNA, small inhibitory RNA and microRNA. In some embodiments, the agent that inhibits bZIP60 expression is dsRNA. In some embodiments, the viral infection is due to a positive-sense ssRNA virus, such as a member of the Flexiviridae family.

Also provided are methods of treating, preventing or slowing viral infection in a plant. The methods comprise a step of inhibiting activity of a bZIP60 protein in plant cells of said plant.

Transgenic plants that are genetically engineered to have impaired bZIP60 activity are also provided. In some embodiments, the genetically engineered plants lack a bZIP60 gene. In some embodiments, the transgenic plant is genetically engineered to contain and express i) a mutant bZIP60 gene that is expressed at a level that is lower than a comparable wild type bZIP60 gene; or ii) a mutant bZIP60 gene that expresses a bZIP60 protein that has a level of activity that is lower than that of a comparable wild-type bZIP60 protein. In other embodiments, the transgenic plant is genetically engineered to contain and express an at least one agent that inhibits: i) expression of a bZIP60 gene; or ii) activity of a bZIP60 gene product.

Also provided are methods of inducing the unfolded protein response (UPR) in a cell. The methods comprise the step of providing to the cell in a quantity of TGBp3 sufficient to induce said UPR in said cell. In some embodiments, the TGBp3 is provided to endoplasmic reticulum (ER) of said cell. In some embodiments, the cell is a cancer cell and induction of UPR results in apoptosis of said cell. In other embodiments, the cell is a damaged neural cell and induction of UPR triggers pro-survival pathways in said cell. The cell in which UPR is induced may be in a mammal.

### BRIEF DESCRIPTION OF THE FIGURES

FIG. 1. Boxplots representing qRT-PCR analysis transcript levels in PVX infected N. benthamiana leaves. Genes are indicated above each graph. Box plots represent the range of values obtained for 20 samples and the variability of gene expression. The boundaries of each box represent the lower 25th and upper 75th percentiles, and the horizontal line within the box represents the median value (i.e. 50th percentile). The spacing of components within the box indicates the degree of dispersal, or skewness, in the data. The lines at the top and bottom of the box (whiskers) represent the sample minimum and maximum. Longer lines at the top indicate a positive skewness. Outliers are indicated by 3. Kruskal-Wallis test of the equality of medians reported P values as follows: for bZIP60, P, 0.0001; for BiP, P, 0.001; for PDI, P=0.007; for CAM, P=0.0143; for CRT, P=0.007; and for SKP1, P, 0.0001.

FIG. 2A-C. TGBp3 induction of BiP, CAM, CRT, PDI, SKP1, and bZIP60 transcripts following agro-infiltration. A, Diagrammatic representation of constructs used in this study. The black arrows indicate the CaMV 35S promoter, and the light gray arrow indicates the NOS promoter. The boxes represent open reading frames. The name for each construct is listed on the right. The gray bars indicate myc or His tags. B, Immunoblots containing protein extracts from N. benthamiana leaves that were infiltrated with buffer (0; lane 1) or A. tumefaciens containing PVX-GFP(P), mycRep (myc-R), mycTGBp2 (myc-p2), TGBp3His (p3His), TGBp1 (p1), and CP. The immunoblot at the bottom shows PVX-GFP and PVX-p3H is. The latter contains a His tag fused to TGBp3, and immunoblotting was carried out using CP antisera. The immunoblot shows that CP levels are comparable in systemic tissues at 7 dpi, indicating that the His tag is not deleterious to virus accumulation. The antisera used for protein detection are identified below the blots. The PVX used in these experiments has the His tag fused to TGBp3; therefore, H is antisera can detect TGBp3 in the PVX genome. C, Leaves were agroinfiltrated, total RNA was extracted at 2 or 5 dpi, and qRT-PCR was carried out. Values represent averages of three replicate samples FIGS. 3A and B. Induction following delivery of TGBp3 to Arabidopsis and using alternative ectopic promoters. The top of each panel shows an immunoblot probed with His antisera. In A, the immunoblot contains protein extracts from Arabidopsis leaves that were infiltrated with buffer (M), A. tumefaciens only (O), or A. tumefaciens containing plasmids expressing TGBp3-His. In B, the immunoblot contains protein extracts from N. benthamiana leaves that were infiltrated with A. tumefaciens containing TGBp3 fused to either the NOS promoter (lanes 2-5) or the CaMV 35S promoter (lanes 6-9). These immunoblots verify protein expression in planta. The Coomassie blue-stained gel located below each immunoblot shows equal sample loading on the gel. A, Arabidopsis leaves were infiltrated with buffer (mock), A. tumefaciens only (Agro), or A. tumefaciens expressing 35S-TGBp3. Total RNA was extracted at 2 or 5 dpi, and qRT-PCR was carried out. The average of three replicate samples is represented by each bar. ANOVA was used to verify that TGBp3 induced higher levels of host transcripts than other treatments at that time point (P, 0.05). B, N. benthamiana leaves were infiltrated with buffer (mock) or dilutions of A. tumefaciens containing TGBp3 fused to either the CaMV 35S or NOS promoter.

FIG. 4. Effects of TRV-bZIP60 on expression of bZIP60, BiP, and SKP 1. Semiquantitative RT-PCR was conducted to verify silencing following TRV-VIGS treatment. The name of the gene analyzed by RTPCR is listed at the top left of each panel. The sizes in by of the DNA ladder (L) are indicated on the left. The bottom of each lane indicates the number of PCR cycles performed. PCR bands representing bZIP60, SKP1, and actin (internal control) after 25 cycles are shown for healthy and TRV-treated samples. Below actin are gel panels showing the outcomes of semiquantitative RT-PCR detecting BiP or SKP 1 on bZIP60— silenced plants. Bands representing BiPare not seen until 45 cycles, and SKP1 bands appear at approximately 35 cycles.

FIGS. 5A and B. TRV-VIGS silenced N. benthanmiana plants were inoculated with PVX-GFP. A, Images of systemic PVX-GFP infection at 7 dpi using a handheld UV lamp. Some plants were pretreated with TRV, TRVbZIP60, or TRV-SKP1 and then with PVX-GFP at 14 d following TRV delivery. The insets show bZIP60-silenced plants with PVX-GFP fluorescence in systemic leaves at 9 dpi. B, Immunoblot analysis confirms PVX CP in infected plants at 7 dpi. Treatment with buffer (O), TRVempty vector, TRV-bZIP60, or TRV-SKP1 is indicated above each pair of lanes. The Coomassie blue-stained gel below the immunoblot shows equal sample loading. C, Northern-blot analysis of BY-2 protoplasts at 36 hpi following transfection with PVX-GFP(P) transcripts and dsRNAs used to knock down NbSKP1 or bZIP60 expression. The top of each lane indicates BY-2 protoplasts that are untreated (O), treated with PVX or PVX plus dsRNAs, or treated with dsRNAs alone. Labels on the right indicate RNA probe. An ethidium bromide-stained gel image of rRNA is included below each northern. dsRNAs successfully knocked down SKP1 and bZIP60 expression in BY-2 protoplasts. PVX-GFP accumulation was limited in bZIP60-silenced protoplasts but not in SKP1-silenced protoplasts.

FIGS. 6A and B. Immunoblot analysis following agro-infiltration with combinations of plasmids expressing TGBp3His, mycTGBp2, mycRep. A, N. benthamiana leaves infiltrated with a suspension of A. tumefaciens containing an empty vector (Agro), TGBp3His, mycTGBp2, or mycTGBp2 plus TGBp3H is. The top panel shows an immunoblot probed with myc antiserum. The second panel shows an immunoblot probed with His antiserum. The Coomassie blue-stained gel verifies equal loading of samples. B, N. benthamiana leaves infiltrated with a suspension of A. tumefaciens containing an empty vector, TGBp3His, mycRep, or mycRep plus TGBp3H is. The immunoblot in the top panel was probed with myc antiserum and the second panel was probed with His antiserum. The Coomassie blue-stained gel shows equal loading of samples.

FIG. 7A-C. Overexpression of BiP alleviates TGBp3-induced cell death. A, N. benthamiana leaves infiltrated with A. tumefaciens alone, containing a GUS construct, or TGBp3 fused to CaMV 35S promoter. The bright-field images show necrotic flecks. Bars=50 B, Immunoblot detection of BiP in leaf extracts at 2 d post infiltration. The image shows bright bands corresponding to immunodetected proteins. Films were scanned, and reverse images were recorded for high-resolution visualization of bands. The Coomassie blue-stained membrane shows equal amounts of protein loaded in each lane. C, N. benthamiana leaves infiltrated with A. tumefaciens listed on the left of each pair of panels. The left column shows necrosis seen under a UV lamp, and the right column shows similar tissues following treatment with H2DCFDA stain, which detects ROS activity. Bars=50 μm.

FIGS. 8A and B. ER stress pathways in A, mammalian and B, plant systems.

FIG. 9. Genes identified by microarray analysis to be induced by PVX infection, and examined by qRT-PCR analysis.

FIG. 10A-D. A, nucleic acid sequence of gene encoding the Arabidopsis bZIP60 gene (SEQ ID NO: 23); B, protein product of the bZIP60 gene (SEQ ID NO: 24); C, amino acid sequence of PVX TGBp3 (SEQ ID NO: 25); D, cDNA sequence encoding PVX TGBp3 (SEQ ID NO: 26).

### DETAILED DESCRIPTION

A first aspect of the present disclosure involves manipulation of expression of bZIP60. Thus, in some embodiments, the present disclosure provides methods to decrease, prevent, or inhibit the expression of the bZIP60 gene in plants of interest. Those of skill in the art will recognize that attenuation/modulation of the level of expression of the bZIP60 gene may be accomplished by any of several methods, e.g. by mutating sections of the genome that are responsible for gene transcription, by modifying the gene that is transcribed and translated, etc. Such modifications may be accomplished by adding exogenous materials (e.g. RNA, DNA, proteins, etc.) to a wild type plant or plant cell. Alternatively, such modifications may be accomplished by transforming wild type plants or plant cells via genetic engineering. Methods for carrying out such modifications are discussed below. As will be recognized by those of skill in the art, some methodology is relevant for one or both of these strategies. The decreased level of expression of bZIP60 may be any that is desired, e.g. from 0% (i.e. no mRNA is transcribed from the gene, or no mRNA is translated into protein) to any other level, e.g. about 10, 20, 39, 40, 50, 60, 70, 80, or 90% or more, of the level that is observed in a comparable wild type plant. Those of skill in the art are familiar with such comparisons, e.g. between unmodified “wild type” expression levels, which may be referred to as controls levels, and the level of expression in a plant in which expression has been attenuated. In some embodiments, no bZIP60 expression is present; in other embodiments, a lesser amount of expression is present in plants whose expression level has been modified. “Wild type” (abbreviated “wt”) refers to the phenotype of the typical form of a species as it occurs in nature.

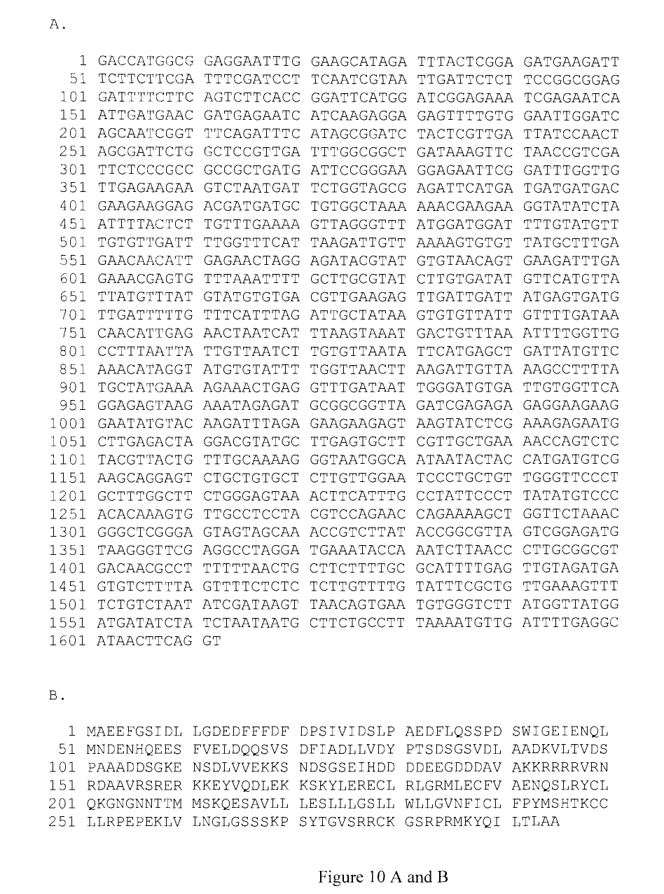

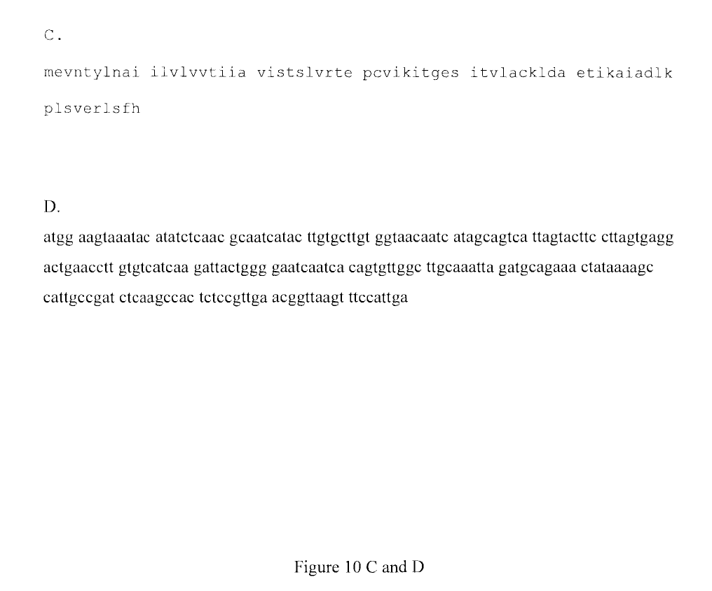

The bZIP60 gene from Potato virus X (PVX) is one exemplary sequence that may be targeted for silencing in the practice of the present disclosure. This gene is also known in the art as “ATBZIP60” and “basic region/leucine zipper motif 60”. The nucleotide sequence of bZIP60 is shown in FIG. 10A and the polypeptide that is translated therefrom is shown in FIG. 10B (see The Arabidopsis Information Resource (TAIR) Protein website, entry AT1G42990.1). However, those of skill in the art will recognize that other alleles, variants and homologs of this gene (and protein) are present in Arabidposis and many other plants which may also be silenced or inhibited as described herein for the same or a similar purpose, for example, at the level of the gene, or at the level of mRNA, or at the level of cDNA. In addition, the protein product may also be inactivated. In some embodiments, the bZIP60 gene is expressed but the gene product (typically a protein or polypeptide) is less active than a gene product from a comparable wild type plant. The level of activity may be any that is desired, e.g. from 0% (i.e. the protein displays no detectable activity) to any other level, e.g. about 10, 20, 39, 40, 50, 60, 70, 80, or 90% or more, of the level of activity that is observed in a comparable wild type plant. In some embodiments, the activity of the polypeptide may be reduced or eliminated by disrupting the gene encoding the polypeptide. The present disclosure encompasses mutagenized plants that carry mutations in such genes, where the mutations reduce expression of the gene or inhibit the activity of the encoded polypeptide.

In other embodiments, an active form of bZIP60 protein (polypeptide) is expressed, but the activity is abolished or decreased by providing to the plant a chemical compound that interferes with the protein's activity.

Thus, many methods may be used to reduce or eliminate the activity of a bZIP60 polypeptide. In addition, more than one method may be used to reduce the activity of a single bZIP60 polypeptide. Non-limiting examples of methods of reducing or eliminating the expression of fully active, wild type bZIP60 polypeptides are given below.

### 1. Polynucleotide-Based Methods:

In some embodiments of the present disclosure, a plant is transformed with an expression cassette that is capable of expressing a polynucleotide that inhibits the expression of a polypeptide of the present disclosure. Expression of the polynucleotide may be inducible, e.g. may occur as the result of a stimulus such as the presence of a virus or expression of a viral protein. The term “expression” as used herein refers to the biosynthesis of a gene product, including the transcription and/or translation of said gene product. For example, for the purposes of the present disclosure, an expression cassette capable of expressing a polynucleotide that inhibits the expression of at least one bZIP60 polypeptide is an expression cassette capable of producing an RNA molecule that inhibits the transcription and/or translation of at least one bZIP60 polypeptide. The “expression” or “production” of a protein or polypeptide from a DNA molecule refers to the transcription and translation of the coding sequence to produce the protein or polypeptide, while the “expression” or “production” of a protein or polypeptide from an RNA molecule refers to the translation of the RNA coding sequence to produce the protein or polypeptide.

Examples of polynucleotides that inhibit the expression of an bZIP60 polypeptide are given below.

i. Sense Suppression/Cosuppression

In some embodiments of the present disclosure, inhibition of the expression of the polypeptide of interest may be obtained by sense suppression or co-suppression. For co-suppression, an expression cassette is designed to express an RNA molecule corresponding to all or part of a messenger RNA encoding a polypeptide in the “sense” orientation. Over expression of the RNA molecule can result in reduced expression of the native gene. Accordingly, multiple plant lines transformed with the cosuppression expression cassette are screened to identify those that show the greatest inhibition of polypeptide expression.

The polynucleotide used for co-suppression may correspond to all or part of the sequence encoding the polypeptide, all or part of the 5′ and/or 3′ untranslated region of the polypeptide transcript or all or part of both the coding sequence and the untranslated regions of a transcript encoding the polypeptide. In some embodiments where the polynucleotide comprises all or part of the coding region for the polypeptide, the expression cassette is designed to eliminate the start codon of the polynucleotide so that no protein product will be translated.

Co-suppression may be used to inhibit the expression of plant genes to produce plants having undetectable protein levels for the proteins encoded by these genes. See, for example, Broin, et al., (2002) Plant Cell 14:1417-1432. Cosuppression may also be used to inhibit the expression of multiple proteins in the same plant. See, for example, U.S. Pat. No. 5,942,657. Methods for using cosuppression to inhibit the expression of endogenous genes in plants are described in Flavell, et al., (1994) Proc. Natl. Acad. Sci. USA 91:3490-3496; Jorgensen, et al., (1996) Plant Mol. Biol. 31:957-973; Johansen and Carrington (2001) Plant Physiol. 126:930-938; Broin, et al., (2002) Plant Cell 14:1417-1432; Stoutjesdijk, et al., (2002) Plant Physiol. 129:1723-1731; Yu, et al., (2003) Phytochemistry 63:753-763 and U.S. Pat. Nos. 5,034,323, 5,283,184 and 5,942,657; each of which is herein incorporated by reference. The efficiency of cosuppression may be increased by including a poly-dT region in the expression cassette at a position 3′ to the sense sequence and 5′ of the polyadenylation signal. See, US Patent Application Publication Number 2002/0048814, herein incorporated by reference. Typically, such a nucleotide sequence has substantial sequence identity to the sequence of the transcript of the endogenous gene, optimally greater than about 65% sequence identity, more optimally greater than about 85% sequence identity, most optimally greater than about 95% sequence identity. See, U.S. Pat. Nos. 5,283,184 and 5,034,323, herein incorporated by reference.

ii. Antisense Suppression

In some embodiments of the present disclosure, inhibition of the expression of the bZIP60 polypeptide may be obtained by antisense suppression. For antisense suppression, the expression cassette is designed to express an RNA molecule complementary to all or part of a messenger RNA encoding the bZIP60 polypeptide. Over expression of the antisense RNA molecule can result in reduced expression of the native gene. Accordingly, multiple plant lines transformed with the antisense suppression expression cassette are screened to identify those that show the greatest inhibition of polypeptide expression.

The polynucleotide for use in antisense suppression may correspond to all or part of the complement of the sequence encoding the bZIP60 polypeptide, all or part of the complement of the 5′ and/or 3′ untranslated region of the bZIP60 transcript or all or part of the complement of both the coding sequence and the untranslated regions of a transcript encoding the bZIP60 polypeptide. In addition, the antisense polynucleotide may be fully complementary (i.e., 100% identical to the complement of the target sequence) or partially complementary (i.e., less than 100% identical to the complement of the target sequence) to the target sequence. Antisense suppression may be used to inhibit the expression of multiple proteins in the same plant. See, for example, U.S. Pat. No. 5,942,657. Furthermore, portions of the antisense nucleotides may be used to disrupt the expression of the target gene. Generally, sequences of at least 50 nucleotides, 100 nucleotides, 200 nucleotides, 300, 400, 450, 500, 550 or greater may be used. Methods for using antisense suppression to inhibit the expression of endogenous genes in plants are described, for example, in Liu, et al., (2002) Plant Physiol. 129:1732-1743 and U.S. Pat. Nos. 5,759,829 and 5,942,657, each of which is herein incorporated by reference. Efficiency of antisense suppression may be increased by including a poly-dT region in the expression cassette at a position 3′ to the antisense sequence and 5′ of the polyadenylation signal. See, US Patent Application Publication Number 2002/0048814, herein incorporated by reference.

iii. Double-Stranded RNA Interference

In some embodiments of the present disclosure, inhibition of the expression of a bZIP60 polypeptide may be obtained by double-stranded RNA (dsRNA) interference. For dsRNA interference, a sense RNA molecule like that described above for cosuppression and an antisense RNA molecule that is fully or partially complementary to the sense RNA molecule are expressed in the same cell, resulting in inhibition of the expression of the corresponding endogenous messenger RNA.

Expression of the sense and antisense molecules can be accomplished by designing the expression cassette to comprise both a sense sequence and an antisense sequence. Alternatively, separate expression cassettes may be used for the sense and antisense sequences. Multiple plant lines transformed with the dsRNA interference expression cassette or expression cassettes are then screened to identify plant lines that show the greatest inhibition of the polypeptide expression. Methods for using dsRNA interference to inhibit the expression of endogenous plant genes are described in Waterhouse, et al., (1998) Proc. Natl. Acad. Sci. USA 95:13959-13964, Liu, et al., (2002) Plant Physiol. 129:1732-1743 and WO 99/49029, WO 99/53050, WO 99/61631 and WO 00/49035, each of which is herein incorporated by reference.

iv. Hairpin RNA Interference and Intron-Containing Hairpin RNA Interference

In some embodiments of the present disclosure, inhibition of the expression of a bZIP60 polypeptide may be obtained by hairpin RNA (hpRNA) interference or intron-containing hairpin RNA (ihpRNA) interference. These methods are highly efficient at inhibiting the expression of endogenous genes. See, Waterhouse and Helliwell, (2003) Nat. Rev. Genet. 4:29-38 and the references cited therein.

For hpRNA interference, the expression cassette is designed to express an RNA molecule that hybridizes with itself to form a hairpin structure that comprises a single-stranded loop region and a base-paired stem. The base-paired stem region comprises a sense sequence corresponding to all or part of the endogenous messenger RNA encoding the gene whose expression is to be inhibited and an antisense sequence that is fully or partially complementary to the sense sequence. Alternatively, the base-paired stem region may correspond to a portion of a promoter sequence controlling expression of the gene to be inhibited. Thus, the base-paired stem region of the molecule generally determines the specificity of the RNA interference. hpRNA molecules are highly efficient at inhibiting the expression of endogenous genes and the RNA interference they induce is inherited by subsequent generations of plants. See, for example, Chuang and Meyerowitz, (2000) Proc. Natl. Acad. Sci. USA 97:4985-4990; Stoutjesdijk, et al., (2002) Plant Physiol. 129:1723-1731 and Waterhouse and Helliwell, (2003) Nat. Rev. Genet. 4:29-38. Methods for using hpRNA interference to inhibit or silence the expression of genes are described, for example, in Chuang and Meyerowitz, (2000) Proc. Natl. Acad. Sci. USA 97:4985-4990; Stoutjesdijk, et al., (2002) Plant Physiol. 129:1723-1731; Waterhouse and Helliwell, (2003) Nat. Rev. Genet. 4:29-38; Pandolfini, et al., BMC Biotechnology 3:7 and US Patent Application Publication Number 2003/0175965, each of which is herein incorporated by reference. A transient assay for the efficiency of hpRNA constructs to silence gene expression in vivo has been described by Panstruga, et al., (2003) Mol. Biol. Rep. 30:135-140, herein incorporated by reference.

For ihpRNA, the interfering molecules have the same general structure as for hpRNA, but the RNA molecule additionally comprises an intron that is capable of being spliced in the cell in which the ihpRNA is expressed. The use of an intron minimizes the size of the loop in the hairpin RNA molecule following splicing, and this increases the efficiency of interference. See, for example, Smith, et al., (2000) Nature 407:319-320. In fact, Smith, et al., show 100% suppression of endogenous gene expression using ihpRNA-mediated interference. Methods for using ihpRNA interference to inhibit the expression of endogenous plant genes are described, for example, in Smith, et al., (2000) Nature 407:319-320; Wesley, et al., (2001) Plant J. 27:581-590; Wang and Waterhouse, (2001) Curr. Opin. Plant Biol. 5:146-150; Waterhouse and Helliwell, (2003) Nat. Rev. Genet. 4:29-38; Helliwell and Waterhouse, (2003) Methods 30:289-295, and US Patent Application Publication Number 2003/0180945, each of which is herein incorporated by reference.

The expression cassette for hpRNA interference may also be designed such that the sense sequence and the antisense sequence do not correspond to an endogenous RNA. In this embodiment, the sense and antisense sequence flank a loop sequence that comprises a nucleotide sequence corresponding to all or part of the endogenous messenger RNA of the target gene. Thus, it is the loop region that determines the specificity of the RNA interference. See, for example, WO 02/00904, Mette, et al., (2000) EMBO J. 19:5194-5201; Matzke, et al., (2001) Curr. Opin. Genet. Devel. 11:221-227; Scheid, et al., (2002) Proc. Natl. Acad. Sci., USA 99:13659-13662; Aufsaftz, et al., (2002) Proc. Natl. Acad. Sci. 99(4):16499-16506; Sijen, et al., Curr. Biol. (2001) 11:436-440), herein incorporated by reference.

v. Amplicon-Mediated Interference

Amplicon expression cassettes comprise a plant virus-derived sequence that contains all or part of the target gene (e.g. the bZIP60 gene) but generally not all of the genes of the native virus. The viral sequences present in the transcription product of the expression cassette allow the transcription product to direct its own replication. The transcripts produced by the amplicon may be either sense or antisense relative to the target sequence (i.e., the messenger RNA for the bZIP60 polypeptide). Methods of using amplicons to inhibit the expression of endogenous plant genes are described, for example, in Angell and Baulcombe, (1997) EMBO J. 16:3675-3684, Angell and Baulcombe, (1999) Plant J. 20:357-362 and U.S. Pat. No. 6,646,805, each of which is herein incorporated by reference.

vi. Ribozymes

In some embodiments, the polynucleotide expressed by the expression cassette of the present disclosure is catalytic RNA or has ribozyme activity specific for the messenger RNA of the bZIP60 polypeptide. Thus, the polynucleotide causes the degradation of the endogenous messenger RNA, resulting in reduced expression of the polypeptide. This method is described, for example, in U.S. Pat. No. 4,987,071, herein incorporated by reference.

vii. Small Interfering RNA or Micro RNA

In some embodiments of the present disclosure, inhibition of the expression of a bZIP60 polypeptide may be obtained by RNA interference by expression of a gene encoding a micro RNA (miRNA). miRNAs are regulatory agents consisting of about 22 ribonucleotides. miRNA are highly efficient at inhibiting the expression of endogenous genes. See, for example, Javier, et al., (2003) Nature 425:257-263, herein incorporated by reference.

For miRNA interference, the expression cassette is designed to express an RNA molecule that is modeled on an endogenous miRNA gene. The miRNA gene encodes an RNA that fauns a hairpin structure containing a 22-nucleotide sequence that is complementary to another endogenous gene (target sequence). For suppression of bZIP60 expression, the 22-nucleotide sequence is selected from a bZIP60 transcript sequence and contains 22 nucleotides of said bZIP60 sequence in sense orientation and 21 nucleotides of a corresponding antisense sequence that is complementary to the sense sequence. miRNA molecules are highly efficient at inhibiting the expression of endogenous genes and the RNA interference they induce is inherited by subsequent generations of plants.

### 2. Polypeptide-Based Inhibition of Gene Expression

In some embodiments, the polynucleotide encodes a zinc finger protein that binds to a gene encoding a polypeptide of interest, resulting in reduced expression of the gene. In particular embodiments, the zinc finger protein binds to a regulatory region of a bZIP60 gene. In other embodiments, the zinc finger protein binds to a messenger RNA encoding the polypeptide and prevents its translation. Methods of selecting sites for targeting by zinc finger proteins have been described, for example, in U.S. Pat. No. 6,453,242 and methods for using zinc finger proteins to inhibit the expression of genes in plants are described, for example, in US Patent Application Publication Number 2003/0037355, each of which is herein incorporated by reference.

### 3. Polypeptide-Based Inhibition of Protein Activity

In some embodiments of the present disclosure, the polynucleotide encodes an antibody that binds to at least one polypeptide of interest and reduces its activity. In another embodiment, the binding of the antibody results in increased turnover of the antibody-polyupeptide complex by cellular quality control mechanisms. The expression of antibodies in plant cells and the inhibition of molecular pathways by expression and binding of antibodies to proteins in plant cells are well known in the art. See, for example, Conrad and Sonnewald, (2003) Nature Biotech. 21:35-36, incorporated herein by reference.

### 4. Gene Disruption

In some embodiments of the present disclosure, the activity of a polypeptide of interest is reduced or eliminated by disrupting the gene encoding the polypeptide. The gene encoding the polypeptide may be disrupted by any method known in the art. For example, in one embodiment, the gene is disrupted by transposon tagging. In another embodiment, the gene is disrupted by mutagenizing plants using random or targeted mutagenesis, and selecting for plants that have the desired trait.

i. Transposon Tagging

In one embodiment of the present disclosure, transposon tagging is used to reduce or eliminate the activity of one or more polypeptides of interest. Transposon tagging comprises inserting a transposon within an endogenous gene to reduce or eliminate expression of the polypeptide.

In this embodiment, the expression of one or more polypeptides of interest is reduced or eliminated by inserting a transposon within a regulatory region or coding region of the gene encoding the polypeptide. A transposon that is within an exon, intron, 5′ or 3′ untranslated sequence, a promoter or any other regulatory sequence of a bZIP60 gene may be used to reduce or eliminate the expression and/or activity of the encoded polypeptide.

Methods for the transposon tagging of specific genes in plants are well known in the art. See, for example, Maes, et al., (1999) Trends Plant Sci. 4:90-96; Dharmapuri and Sonti, (1999) FEMS Microbiol. Lett. 179:53-59; Meissner, et al., (2000) Plant J. 22:265-274; Phogat, et al., (2000) J. Biosci. 25:57-63; Walbot, (2000) Curr. Opin. Plant Biol. 2:103-107; Gai, et al., (2000) Nucleic Acids Res. 28:94-96; Fitzmaurice, et al., (1999) Genetics 153:1919-1928). In addition, the TUSC process for selecting Mu insertions in selected genes has been described in Bensen, et al., (1995) Plant Cell 7:75-84; Mena, et al., (1996) Science 274:1537-1540 and U.S. Pat. No. 5,962,764, each of which is herein incorporated by reference.

### Additional Methods

Additional methods for decreasing or eliminating the expression of endogenous genes in plants are also known in the art and can be similarly applied to the present disclosure. These methods include other forms of mutagenesis, such as ethyl methanesulfonate-induced mutagenesis, deletion mutagenesis and fast neutron deletion mutagenesis used in a reverse genetics sense (with PCR) to identify plant lines in which the endogenous gene has been deleted. For examples of these methods see, Ohshima, et al., (1998) Virology 243:472-481; Okubara, et al., (1994) Genetics 137:867-874 and Quesada, et al., (2000) Genetics 154:421-436, each of which is herein incorporated by reference. In addition, a fast and automatable method for screening for chemically induced mutations, TILLING (Targeting Induced Local Lesions In Genomes), using denaturing HPLC or selective endonuclease digestion of selected PCR products is also applicable to the present disclosure. See, McCallum, et al., (2000) Nat. Biotechnol. 18:455-457, herein incorporated by reference.

Mutations that impact gene expression or that interfere with the function of the encoded protein are well known in the art. Insertional mutations in gene exons usually result in null-mutants. Mutations in conserved residues are particularly effective in inhibiting the activity of the encoded protein. In another embodiment of the present disclosure, dominant mutants can be used to trigger RNA silencing due to gene inversion and recombination of a duplicated gene locus. See, for example, Kusaba, et al., (2003) Plant Cell 15:1455-1467.

In other embodiments of the present disclosure, dominant mutants can be used to trigger RNA silencing due to gene inversion and recombination of a duplicated gene locus. See, for example, Kusaba, et al., (2003) Plant Cell 15:1455-1467. The present disclosure encompasses additional methods for reducing or eliminating the activity of one or more bZIP60 polypeptides. Examples of other methods for altering or mutating a genomic nucleotide sequence in a plant are known in the art and include, but are not limited to, the use of RNA:DNA vectors, RNA:DNA mutational vectors, RNA:DNA repair vectors, mixed-duplex oligonucleotides, self-complementary RNA:DNA oligonucleotides and recombinogenic oligonucleobases. Such vectors and methods of use are known in the art. See, for example, U.S. Pat. Nos. 5,565,350; 5,731,181; 5,756,325; 5,760,012; 5,795,972 and 5,871,984, each of which are herein incorporated by reference. See also, WO 98/49350, WO 99/07865, WO 99/25821 and Beetham, et al., (1999) Proc. Natl. Acad. Sci. USA 96:8774-8778, each of which is herein incorporated by reference.

Issued U.S. Pat. Nos. 8,252,979 (Gupta, et al.), 8,269,065 (Privat, et al.), 8,269,082 (Millar, et al.), 8,273,950 (Lepelley, et al.), 8,252,977 (Tanksley, et al.), 8,247,646 (Guo, et al.) and 8,237,015 (Luo) also provide descriptions of various methods to alter gene expression in plants, and the complete contents of each of these is herein incorporated by referenced in entirety.

### Transgenic Plants

The present disclosure also encompasses transgenic plants that have been genetically manipulated to express forms of bZIP60 that possess a desired level of activity (that is usually lower than wild type activity), to express factors or elements that attenuate the level of activity of bZIP60 or which modulate the expression of the bZIP60 gene. The desired level may be any that is suitable to lower the incidence, severity, duration, or extent of viral infections, or which reduces the plants susceptibility to viral infection, compared to a comparable wild type control. Alternatively, the plants may be genetically engineered to decrease expression in an inducible manner, e.g. in response to a trigger that occurs at the onset of a viral infection (e.g. expression or presence of a viral protein), or an externally applied trigger or stimulus that is employed when the plant is at risk of viral infection or when viral infection is suspected or known to be present. Methods for genetically engineering plant cells and plants are known and are described below.

### Plant Transformation Methods

Numerous methods for introducing foreign genes into plants are known and can be used to insert a bZIP60 polynucleotide into a plant host, including biological and physical plant transformation protocols. See, e.g., Miki, et al., “Procedure for Introducing Foreign DNA into Plants,” in METHODS IN PLANT MOLECULAR BIOLOGY AND BIOTECHNOLOGY, Glick and Thompson, eds., CRC Press, Inc., Boca Raton, pp. 67-88 (1993). The methods chosen vary with the host plant, and include chemical transfection methods such as calcium phosphate, microorganism-mediated gene transfer such as Agrobacterium (Horsch, et al., (1985) Science 227:1229-31), electroporation, micro-injection, and biolistic bombardment.

Expression cassettes and vectors and in vitro culture methods for plant cell or tissue transformation and regeneration of plants are known and available. See, e.g., Gruber, et al., “Vectors for Plant Transformation,” in METHODS IN PLANT MOLECULAR BIOLOGY AND BIOTECHNOLOGY, supra, pp. 89-119.

Isolated polynucleotides or polypeptides may be introduced into the plant by one or more techniques typically used for direct delivery into cells. Such protocols may vary depending on the type of organism, cell, plant or plant cell, i.e., monocot or dicot, targeted for gene modification. Suitable methods of transforming plant cells include microinjection (Crossway, et al., (1986) Biotechniques 4:320-334 and U.S. Pat. No. 6,300,543), electroporation (Riggs, et al., (1986) Proc. Natl. Acad. Sci. USA 83:5602-5606, direct gene transfer (Paszkowski, et al., (1984) EMBO J. 3:2717-2722) and ballistic particle acceleration (see, for example, Sanford, et al., U.S. Pat. No. 4,945,050; WO 91/10725 and McCabe, et al., (1988) Biotechnology 6:923-926). Also see, Tomes, et al., Direct DNA Transfer into Intact Plant Cells Via Microprojectile Bombardment. pp. 197-213 in Plant Cell, Tissue and Organ Culture, Fundamental Methods. eds. O. L. Gamborg & G. C. Phillips. Springer-Verlag Berlin Heidelberg New York, 1995; U.S. Pat. No. 5,736,369 (meristem); Weissinger, et al., (1988) Ann. Rev. Genet. 22:421-477; Sanford, et al., (1987) Particulate Science and Technology 5:27-37 (onion); Christou, et al., (1988) Plant Physiol. 87:671-674 (soybean); Datta, et al., (1990) Biotechnology 8:736-740 (rice); Klein, et al., (1988) Proc. Natl. Acad. Sci. USA 85:4305-4309 (maize); Klein, et al., (1988) Biotechnology 6:559-563 (maize); WO 91/10725 (maize); Klein, et al., (1988) Plant Physiol. 91:440-444 (maize); Fromm, et al., (1990) Biotechnology 8:833-839 and Gordon-Kamm, et al., (1990) Plant Cell 2:603-618 (maize); Hooydaas-Van Slogteren and Hooykaas (1984) Nature (London) 311:763-764; Bytebier, et al., (1987) Proc. Natl. Acad. Sci. USA 84:5345-5349 (Liliaceae); De Wet, et al., (1985) In The Experimental Manipulation of Ovule Tissues, ed. G. P. Chapman, et al., pp. 197-209 Longman, N.Y. (pollen); Kaeppler, et al., (1990) Plant Cell Reports 9:415-418; and Kaeppler, et al., (1992) Theor. Appl. Genet. 84:560-566 (whisker-mediated transformation); U.S. Pat. No. 5,693,512 (sonication); D'Halluin, et al., (1992) Plant Cell 4:1495-1505 (electroporation); Li, et al., (1993) Plant Cell Reports 12:250-255 and Christou and Ford, (1995) Annals of Botany 75:407-413 (rice); Osjoda, et al., (1996) Nature Biotech. 14:745-750; Agrobacterium mediated maize transformation (U.S. Pat. No. 5,981,840); silicon carbide whisker methods (Frame, et al., (1994) Plant J. 6:941-948); laser methods (Guo, et al., (1995) Physiologia Plantarum 93:19-24); sonication methods (Bao, et al., (1997) Ultrasound in Medicine & Biology 23:953-959; Finer and Finer, (2000) Lett Appl Microbiol. 30:406-10; Amoah, et al., (2001) J Exp Bot 52:1135-42); polyethylene glycol methods (Krens, et al., (1982) Nature 296:72-77); protoplasts of monocot and dicot cells can be transformed using electroporation (Fromm, et al., (1985) Proc. Natl. Acad. Sci. USA 82:5824-5828) and microinjection (Crossway, et al., (1986) Mol. Gen. Genet. 202:179-185), all of which are herein incorporated by reference.

### Agrobacterium-Mediated Transformation

The most widely utilized method for introducing an expression vector into plants is based on the natural transformation system of Agrobacterium. A. tumefaciens and A. rhizogenes are plant pathogenic soil bacteria, which genetically transform plant cells. The Ti and Ri plasmids of A. tumefaciens and A. rhizogenes, respectively, carry genes responsible for genetic transformation of plants. See, e.g., Kado, (1991) Crit. Rev. Plant Sci. 10:1. Descriptions of the Agrobacterium vector systems and methods for Agrobacterium-mediated gene transfer are provided in Gruber, et al., supra; Miki, et al., supra and Moloney, et al., (1989) Plant Cell Reports 8:238.

Similarly, the gene can be inserted into the T-DNA region of a Ti or Ri plasmid derived from A. tumefaciens or A. rhizogenes, respectively. Thus, expression cassettes can be constructed as above, using these plasmids. Many control sequences are known which when coupled to a heterologous coding sequence and transformed into a host organism show fidelity in gene expression with respect to tissue/organ specificity of the original coding sequence. See, e.g., Benfey and Chua, (1989) Science 244:174-81. Particularly suitable control sequences for use in these plasmids are promoters for constitutive leaf-specific expression of the gene in the various target plants. Other useful control sequences include a promoter and terminator from the nopaline synthase gene (NOS). The NOS promoter and terminator are present in the plasmid pARC2, available from the American Type Culture Collection and designated ATCC 67238. If such a system is used, the virulence (vir) gene from either the Ti or Ri plasmid must also be present, either along with the T-DNA portion, or via a binary system where the vir gene is present on a separate vector. Such systems, vectors for use therein, and methods of transforming plant cells are described in U.S. Pat. No. 4,658,082; U.S. patent application Ser. No. 913,914, filed Oct. 1, 1986, as referenced in U.S. Pat. No. 5,262,306, issued Nov. 16, 1993 and Simpson, et al., (1986) Plant Mol. Biol. 6:403-15 (also referenced in the '306 patent), all incorporated by reference in their entirety.

Once constructed, these plasmids can be placed into A. rhizogenes or A. tumefaciens and these vectors used to transform cells of plant species, which are ordinarily susceptible to Fusarium or Alternaria infection. The selection of either A. tumefaciens or A. rhizogenes will depend on the plant being transformed thereby. In general A. tumefaciens is the preferred organism for transformation. Most dicotyledonous plants, some gymnosperms and a few monocotyledonous plants (e.g., certain members of the Liliales and Arales) are susceptible to infection with A. tumefaciens. A. rhizogenes also has a wide host range, embracing most dicots and some gymnosperms, which includes members of the Leguminosae, Compositae and Chenopodiaceae. Monocot plants can now be transformed with some success. EP Patent Application Number 604 662 A1 discloses a method for transforming monocots using Agrobacterium. EP Application Number 672 752 A1 discloses a method for transforming monocots with Agrobacterium using the scutellum of immature embryos. Ishida, et al., discuss a method for transforming maize by exposing immature embryos to A. tumefaciens (Nature Biotechnology 14:745-50 (1996)).

Once transformed, these cells can be used to regenerate transgenic plants. For example, whole plants can be infected with these vectors by wounding the plant and then introducing the vector into the wound site. Any part of the plant can be wounded, including leaves, stems and roots. Alternatively, plant tissue, in the form of an explant, such as cotyledonary tissue or leaf disks, can be inoculated with these vectors, and cultured under conditions, which promote plant regeneration. Roots or shoots transformed by inoculation of plant tissue with A. rhizogenes or A. tumefaciens, containing the gene coding for the fumonisin degradation enzyme, can be used as a source of plant tissue to regenerate fumonisin-resistant transgenic plants, either via somatic embryogenesis or organogenesis. Examples of such methods for regenerating plant tissue are disclosed in Shahin, (1985) Theor. Appl. Genet. 69:235-40; U.S. Pat. No. 4,658,082; Simpson, et al., supra and U.S. patent application Ser. Nos. 913,913 and 913,914, both filed Oct. 1, 1986, as referenced in U.S. Pat. No. 5,262,306, issued Nov. 16, 1993, the entire disclosures therein incorporated herein by reference.

### Direct Gene Transfer

Despite the fact that the host range for Agrobacterium-mediated transformation is broad, some major cereal crop species and gymnosperms have generally been recalcitrant to this mode of gene transfer, even though some success has recently been achieved in rice (Hiei, et al., (1994) The Plant Journal 6:271-82). Several methods of plant transformation, collectively referred to as direct gene transfer, have been developed as an alternative to Agrobacterium-mediated transformation.

A generally applicable method of plant transformation is microprojectile-mediated transformation, where DNA is carried on the surface of microprojectiles measuring about 1 to 4 μm. The expression vector is introduced into plant tissues with a biolistic device that accelerates the microprojectiles to speeds of 300 to 600 m/s which is sufficient to penetrate the plant cell walls and membranes (Sanford, et al., (1987) Part. Sci. Technol. 5:27; Sanford, (1988) Trends Biotech 6:299; Sanford, (1990) Physiol. Plant 79:206 and Klein, et al., (1992) Biotechnology 10:268).

Another method for physical delivery of DNA to plants is sonication of target cells as described in Zang, et al., (1991) BioTechnology 9:996. Alternatively, liposome or spheroplast fusions have been used to introduce expression vectors into plants. See, e.g., Deshayes, et al., (1985) EMBO J. 4:2731 and Christou, et al., (1987) Proc. Natl. Acad. Sci. USA 84:3962. Direct uptake of DNA into protoplasts using CaCl.sub.2 precipitation, polyvinyl alcohol, or poly-L-ornithine has also been reported. See, e.g., Hain, et al., (1985) Mol. Gen. Genet. 199:161 and Draper, et al., (1982) Plant Cell Physiol. 23:451.

Electroporation of protoplasts and whole cells and tissues has also been described. See, e.g., Donn, et al., (1990) in Abstracts of the VIIth Int'l. Congress on Plant Cell and Tissue Culture IAPTC, A2-38, p. 53; D'Halluin, et al., (1992) Plant Cell 4:1495-505 and Spencer, et al., (1994) Plant Mol. Biol. 24:51-61.

In some embodiments of the present disclosure, the infections that are slowed or prevented in plants are caused by the virus family Flexiviridae. Those of skill in the art will recognize that the Flexiviridae were a new family of viruses in the 2004 classification of viruses, and have since the 2009 classification been split into the three new families Alphaflexiviridae, Betaflexiviridae and Gammaflexiviridae. These have in turn been subsumed under the new order Tymovirales along with the old family Tymoviridae by the International Committee on Taxonomy of Viruses based on molecular phylogenetic systematic analyses of proteins (RNA polymerase and viral coat). The viruses are positive-sense ssRNA viruses, placing them in Group IV of the Baltimore classification. These viruses are filamentous and named for being highly flexible. Members of these families are readily transmitted mechanically and have other vectors of transmission. Species tend to be confined to a single host plant, many species preferring woody hosts, but a diversity of angiosperm hosts are known to the Flexiviridae. Infection of plant cells by any and all members of this group may be prevented, slowed, lessened, etc. by the practice of the present methods.

Exemplary viral infections that may be treated or addressed in this manner include but are not limited to those caused by:

i) the Alphaflexiviridae family, including the following genera: Genus Allexivirus; type species: Shallot virus X; Genus Botrexvirus; Genus Lolavirus; Genus Mandarivirus; type species: Indian citrus ringspot virus; Genus Potexvirus; type species: Potato virus X; Genus Sclerodarnavirus;

ii) the Betaflexiviridae family, including the following genera: Genus Capillovirus; type species: Apple stem grooving virus; Genus Carlavirus; type species: Carnation latent virus; Genus Citrivirus; Genus Foveavirus; type species: Apple stem pitting virus; Genus Tepovirus: type species: Potato virus T; Genus Trichovirus; type species: Apple chlorotic leaf spot virus; Genus Vitivirus; type species: Grape vine virus A; and

iii) the Gammaflexiviridae family, including the following genera: Genus Mycoflexivirus.

Those of skill in the art will recognize that the methods of the present disclosure may be applied to any plant that is susceptible to viral infection, especially to infection by ssRNA viruses. By “plants” we mean living organisms of the kingdom Plantae including such multicellular groups as flowering plants, conifers, ferns and mosses. Exemplary plant types include but are not limited to: Green algae (Chlorophyta, Charophyta); Land plants (embryophytes), including i) Non-vascular land plants (bryophytes) such as Marchantiophyta—liverworts, Anthocerotophyta—hornworts, Bryophyta—mosses, Horneophytopsida; ii) Vascular plants (tracheophytes), Rhyniophyta—rhyniophytes, Zosterophyllophyta—zosterophylls, Lycopodiophyta—clubmosses, Trimerophytophyta—trimerophytes, Pteridophyta—ferns and horsetails, Progymnospermophyta; and Seed plants (spermatophytes) such as Pteridospermatophyta—seed ferns, Pinophyta—conifers, Cycadophyta—cycads, Ginkgophyta—ginkgo, and Gnetophyta.

In addition, other relevant ssRNA viruses which cause infection that can be treated, prevented of lessened by the methods of the present disclosure include but are not limited to: potato viruses A, M, S, T, V and Y; white clover mosaic, narcissus mosaic, potexviruses, carlaviruses, beet necrotic yellow vein furovirus, barley stripe mosaic hordeivirus, other potexviruses, various tymoviruses, carlaviruses, beet necrotic yellow vein furovirus, barley stripe mosaic hordeivirus, potyviruses, barley yellow mosaic virus, as well as ‘picorna-like’, and ‘sindbis-like’ viruses.

Particular plants of interest in the practice of the present disclosure include but are not limited to those of commercial value such as crop plants (soybeans, wheat, corn, rye, flax, oats, vegetables, fruits, berries, tobacco, coffee, grapes, etc.) as well as decorative or ornamental plants. The term “plant” includes all parts of a plant (e.g. stem, leaves, roots, etc.), all life stage of a plant (e.g. seeds, sprouts, developing plants, plants bearing fruits, etc.), as well as individual cells of a plant, e.g. cells isolated in a laboratory setting, cloned plant cells, cells propagated in vitro, etc.

Vectors encoding the suppressing agents described above are also contemplated, as are formulations suitable for delivering the agents to a plant or plant cells.

### II. TGBp3

A second aspect of the present disclosure involves manipulation of expression of TGBp3. TGBp3 proteins (e.g. those encoded by PVX) associate with (reside in, are present in) the ER and induce or mediate ER stress and turnover of viral proteins. Their presence is essential (vital) for virus cell-to-cell spread. Given the conservation of UPR across eukaryotes, TGBp3 (such as PVX TBBp3 and homologues thereof) may be used to elicit the UPR in the cells of an organism in need thereof.

Due to commonalities of the UPR across species, TGBp3 may be employed in non-plant species to induce UPR in order to regulate disease processes and progression. For example, exposing neural cells involved in neurodegenerative diseases or cancer cells to TGBp3 (especially certain cancers that show elevated Bcl-2 expression and are resistant to chemotherapies), alters the regulation of UPR pathways in those cells. As a result, cellular apoptosis and/or oxidative stress responses are induced or altered in the cells. TGBp3 may trigger UPR pathways that are shut down in cancer cells, advantageously leading to cancer cell death. In addition, TGBp3 may trigger pro-survival pathways in neural cells that would lessen the degeneration caused by some diseases.

The delivery of TGBp3 to the cells of an organism in need thereof (e.g. a mammal such as a human, although veterinary applications are also contemplated) can be accomplished by any of the many methods that are known to those of skill in the art. For example, a gene encoding a TGBp3 protein may be delivered via a nucleic acid vector that is known to be used for gene therapy applications such as an adenoviral or retroviral vector, a herpes simplex viral vector, and HIV viral vector, using liposomes, virosomes, etc. Alternatively, such genes may be delivered as “naked” DNA e.g. by methods such as electroporation, sonoporation, the use of a “gene gun”, by magnetofection, etc. Alternatively, in some embodiments, TGBp3 protein may be administered to a subject in need of thereof.

The sequence of PVX TGBp3 protein is depicted in FIG. 10C and the nucleic acid encoding the protein is depicted in FIG. 10D. This protein is also known in the art as “movement protein TGBp3”, “7 kDa protein”, and “triple gene block 3 protein” (GenBank Accession: P17781.1 GI:137213). Those of skill in the art will recognize that similar homologous nucleic acid sequences are present in other viruses and may be used as described herein. Also, variants of these sequences which retain activity may be employed, e.g. sequence encoding a functional gene product such as nucleic acid sequences with at least about 50, 60, 70, 80, 85, 90, or 95% or more (e.g. 96, 97, 98 or 99%), homology may be used, with homology being determined by standard comparative methods known in the art. The present disclosure also encompasses vectors (plasmids, cosmids, viral vectors, etc.) housing the nucleic acid sequences. Similarly, functionally identical equivalents of the protein may also be employed, e.g. those displaying usually at least about 75, 80, 85, 90, 95% or more similarity (identity) with the PVX TGBp3 amino acid sequence, or an equivalent sequence from another virus (i.e. a sequence having the same function). Such equivalents or variants may contain, for example, conservative amino acid substitutions, various deletions or additions or mutations that do not impair activity. Various tags (e.g. His tags) may be added for the purposes of ease of laboratory manipulation of the sequence. Amino acid residues may be added, removed or changed so as to alter or improve, for example, in vivo stability, solubility, etc.

Accordingly, the present disclosure provides compositions for use in delivering TGBp3 to a subject, whether as a nucleic acid or protein. The compositions include one or more substantially purified TGBp3 encoding nucleic acids or TGBp3 proteins, and a pharmacologically suitable carrier. The preparation of such compositions is well known to those of skill in the art. Typically, such compositions are prepared either as liquid solutions or suspensions, however solid forms such as tablets, pills, powders and the like are also contemplated. Solid forms suitable for solution in, or suspension in, liquids prior to administration may also be prepared. The preparation may also be emulsified. The active ingredients may be mixed with excipients which are pharmaceutically acceptable and compatible with the active ingredients. Suitable excipients are, for example, water, saline, dextrose, glycerol, ethanol and the like, or combinations thereof. In addition, the composition may contain minor amounts of auxiliary substances such as wetting or emulsifying agents, pH buffering agents, and the like. In addition, the composition may contain adjuvants. If it is desired to administer an oral form of the composition, various thickeners, flavorings, diluents, emulsifiers, dispersing aids or binders and the like may be added. The composition of the present disclosure may contain any such additional ingredients so as to provide the composition in a form suitable for administration. The final amount of protein or encoding nucleic acid in the formulations may vary. However, in general, the amount in the formulations will be from about 1-99%.

The compositions (preparations) of the present disclosure may be administered by any of the many suitable means which are well known to those of skill in the art, including but not limited to by injection, inhalation, orally, intravaginally, intranasally, by absorption through epithelial or mucocutaneous linings (e.g., nasal, oral, vaginal, rectal, gastrointestinal mucosa, and the like), by ingestion of a food or probiotic product, topically, as eye drops, via sprays, incorporated into dressings or bandages (e.g. lyophilized forms may be included directly in the dressing), etc. In preferred embodiments, the mode of administration is topical or orally or by injection. If by injection, administration may be intravenous, intraperitoneal, intramuscular, subcutaneous, intra-aural, intraarticular, intramammary, intratumoral, and the like. In addition, the compositions may be administered in conjunction with other treatment modalities such as substances that boost the immune system, various chemotherapeutic agents, antibiotic agents, and the like. The amount of TGBp3 that is administered will depend on the species of the subject, as well as other factors such as the illness being treated, gender, size, overall health, age, etc. and is generally determined by a skilled medical practitioner in the course of clinical trials.

Diseases that may be treated in this manner include but are not limited to: various types of cancer (e.g. pancreatic, prostate, breast, cervical, uterine, bone, brain, head or neck cancer, etc.) and various neurodegenerative diseases (e.g. Parkinson's, Alzheimer's, and Huntington's, etc.). Of particular interest are diseases caused by protein misfolding and/or abnormal functioning of protein degradation and turnover.

### EXAMPLES

While viral modification of the ER architecture has been explored in plants, there are no reported studies examining the role for UPR pathways in plant viral disease. Therefore, we decided to investigate whether Potato virus X (PVX) can also modulate UPR signaling pathways to modify the cellular environment, as described for many mammalian viruses. We explored the role of ER stress in PVX pathogenesis because of the broad range of viral proteins that are known to associate with the ER. Given the role of the proteasome in regulating viral protein accumulation and evidence that virus infection leads to expansion of the ER, we hypothesized that PVX infection could cause mild ER stress leading to up-regulation of the UPR. Herein, we provide evidence that PVX TGBp3 up-regulates UPR-related genes, including bZIP60, when it is expressed from the PVX genome or heterologous expression vectors. We investigate the role of ER stress in maintaining persistent virus infection and conclude that the UPR is a contributing factor toward promoting virus spread. We also link bZIP60 to SKP1 and UPR signaling and systemic accumulation of PVX.

As shown in the Examples provided herein, virus induced gene silencing was employed to knock down expression of the bZIP60 gene in plants and, as a result, the number of infection foci on inoculated leaves was reduced and systemic virus accumulation was altered. Silencing bZIP60 led to suppression of BiP and SKP1 transcript levels suggesting that bZIP60 might be an upstream signal transducer, the suppression of which could be used advantageously to slow or prevent the spread of viruses from cell to cell in a host, and thus to prevent or treat viral infections.

In addition, as shown in the Examples provided herein, expression of TGBp3 in plants led to localized necrosis, but co-expression of TGBp3 with BiP abrogated necrosis. This observation likely indicates that UPR induction by BiP alleviates ER stress related cell death. Taken together, PVX TGBp3 induced ER stress leads to upregulation of bZIP60 and UPR related gene expression and appears to be important to regulate cellular cytotoxicity that could otherwise lead to cell death, e.g. if viral proteins reach high levels in the ER. Thus, delivery of TGBp3 to a cell of interest, in particular to the ER of the cell, can be used to control UPR induction, thereby causing cellular apoptosis (e.g. of cancer cells), or cell survival by triggering pro-survival pathways (e.g. of degenerating cells).

### Introduction

Viral interactions with the host endoplasmic reticulum (ER) are central to their life cycle and these contribute to their pathogenesis. While the ER provides a physical scaffold for building viral protein complexes that are essential for pathogenesis, the ER is also involved in signal transduction relating to stress and immunity. Antiviral compounds targeting these signaling events offer a viable strategy for controlling or abrogating virus infection in agriculture. Such compounds may be, for example, synthetic chemical inhibitors, overexpressed genes that can counter the signal transduction events, or small RNAs that can eliminate the expression of ER-related signaling molecules that are essential for pathogenesis.

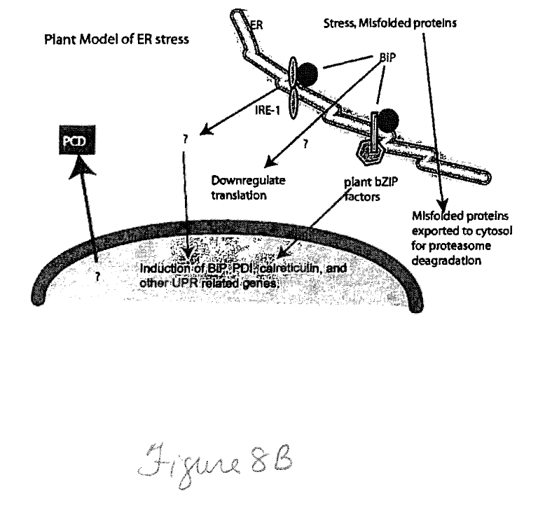

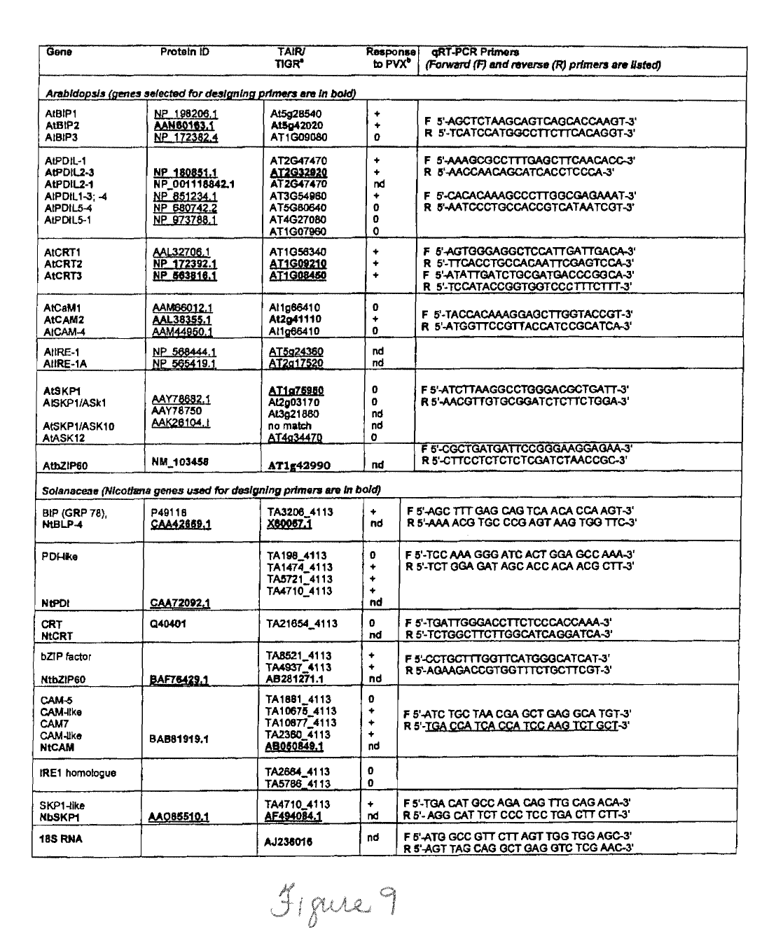

External stimuli such as pathogen invasion, nutrient depletion, or glucose deprivation can exert stress on the ER by causing vigorous protein synthesis, aberrations in Ca2+ or redox regulation, inhibition of protein glycosylation, or protein transfer to the Golgi. These responses increase the levels of misfolded proteins in the ER and trigger the unfolded protein response (UPR). Export of malformed proteins from the ER into the cytosol is followed by degradation via the ubiquitin-proteasome pathway. Thus the purpose of the UPR is to restore normal ER function, relieve stress exerted on the ER, and prevent the cytotoxic impact of malformed proteins (Jelitto-Van Dooren et al., 1999; Xu et al., 2005; Slepak et al., 2007; Urade, 2007; Preston et al., 2009). Many UPR signaling components are conserved among mammals, yeast, and plants although mammals and plants each have additional factors that lead to unique and complex sets of cellular responses (Xu et al., 2005; Zhang and Kaufman, 2006) (FIGS. 8A and 8B). Nutrient depletion or pharmacological agents, such as tunicamycin, have been used to map the plant signaling pathways relating to ER stress and UPR (Williams and Lipkin, 2006).

In both mammals and plants, the UPR mechanism involves increasing synthesis of several ER resident proteins needed to restore proper protein folding such as the ER luminal binding protein (BiP), protein disulfide isomerase (PDI), calreticulin (CRT), and calmodulin (CAM) (FIGS. 8A and 8B) (Navazio et al., 2001; Ellgaard and Helenius, 2003; Oh et al., 2003; Urade, 2007; Seo et al., 2008). In tobacco, NtBLP-4 (the ER luminal binding protein BiP), NtCRT, and NtPDI were specifically upregulated by ER stress inducing compounds (Denecke et al., 1991; Denecke et al., 1995; Iwata and Koizumi, 2005). In fact NtBLP-4 is linked to pro-survival responses in plants and its overexpression alleviates ER stress (Leborgne-Castel et al., 1999). Other plant pro-survival factors include SDF2 which is a target of the UPR and contributes to plant development (Schott et al., 2010).

PVX infection in Nicotiana benthamiana plants leads to increased transcript levels of several stress related host genes including, bZIP60, SKP1, BiP, PDI, CRT, and CAM bZIP60 is a key transcription factor that functions in response to endoplasmic reticulum (ER) stress and induces expression of ER resident chaperones (BiP, PDI, CRT, and CAM). SKP1 is a component of SCF (SKP)-Cullin-F box protein) ubiquitin ligase complexes that target proteins for proteasomal degradation. Expression of PVX TGBp3 from a heterologous vector induces the same set of genes in N. benthamiana and Arabidopsis leaves.

The role of ubiquitin ligase complexes in ER stress as well as in pathogen defense and susceptibility is particularly intriguing, since this study investigates the role of ER stress in virus infection. Without knowing a specific link between the UPR and the proteasome in plants, we chose to examine changes in NbSKP 1 expression following the application of a viral ER stress elicitor in an attempt to link virus induced UPR with at least one component of the SCF complex.

In this Example, we show that silencing bZIP60 eliminated PVX infection in protoplasts. In plants we suppressed bZIP60 by approximately 77% and this slowed down virus infection. We believe that small RNAs could be engineered to improve bZIP60 suppression in a manner that would eliminate PVX infection in plants. This strategy could be applied to a broader range of viruses in Flexiviridae. Since bZIP60 is TGBp3 induced, both genes are central to the control of disease. For biotechnology strategies targeting bZIP60 and/or TGBp3 are effective control measures.

### Upregulation of UPR During PVX Infection

We compared the gene expression profiles obtained using Arabidopsis and potato microarrays, which were reported by Whitham et al., (2003) and Garcia-Marcos et al., (2009), to identify common ER stress regulated genes that are induced by PVX infection. In both investigations, UPR-related ER-resident chaperones as BiP, PDI, and CRT were upregulated, but only the potato microarray detected CAM (FIG. 9). bZIP60, SKP1, but not IRE1 were predicted to be upregulated in the published potato cDNA microarray probed with samples taken from PVX-infected N. benthamiana leaves (Garcia-Marcos et al., 2009). Neither of the ER stress-related sensors bZIP60 nor IRE1 were represented on the Affymetrix Arabidopsis 8K GeneChip oligonucleotide microarray, and therefore, their expression was not determined (FIG. 9). The expression of SKP1 was not reported to be altered in Arabidopsis, although it was represented on the microarray.

To further investigate gene expression associated with the UPR in PVX-infected leaves, qRT-PCR assessment of host transcript accumulation was performed using total RNA isolated from PVX-GFP infected N. benthamiana leaves at 3 and 9 days post inoculation (dpi). Green fluorescent foci appear on the inoculated leaves at 3 dpi. The plants were fully and systemically infected at 9 dpi, although we extracted RNA from the inoculated leaves. Zero dpi represents samples that were harvested just before plants were inoculated with PVX-GFP. Given that the genome sequence for N. benthamiana is incompletely annotated, primers were designed for qRT-PCR based on the sequences of homologs from N. tabacum (NtbZIP60, NtBLP-4, NtCAM, NtCRT, and NtPDI) that have high homology to ESTs identified in the potato microarray (FIGS. 8A and 8B).

Non-parametric analysis was used to describe the distribution of gene expression levels determined by qRT-PCR at 0, 3, or 9 dpi. This method of analysis provides excellent characterization of gene induction when using plant tissues that are not synchronously infected with PVX and/or if host gene expression is transiently altered (Bamunusinghe et al., 2009). Kruskal-Wallis tests (nonparametric analyses of variance) were performed to assess the relationship of time on the various response variables. P-values associated with the tests of equality of medians for each gene examined were less than 0.001, except for CAM whose p-value was 0.014. All p-values indicate that PVX infection caused a significant increase in the expression of each gene over time.

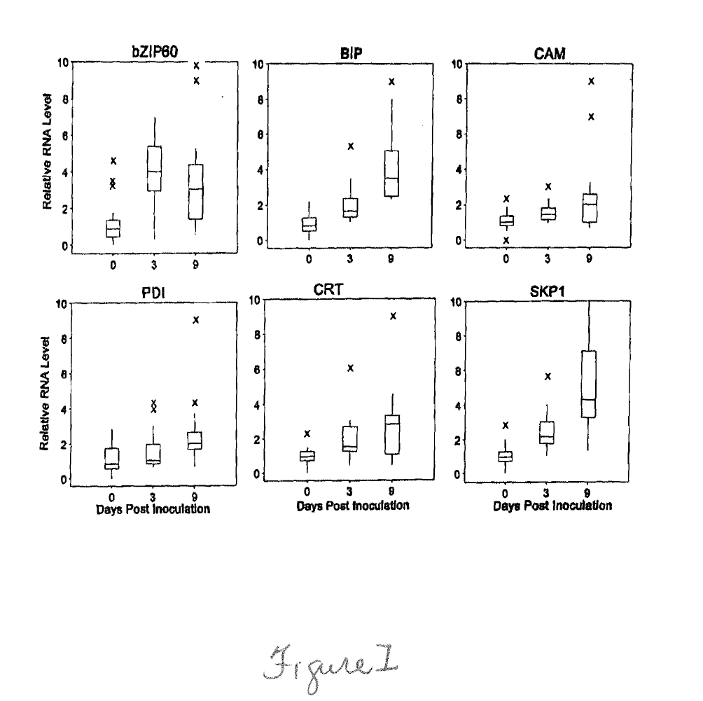

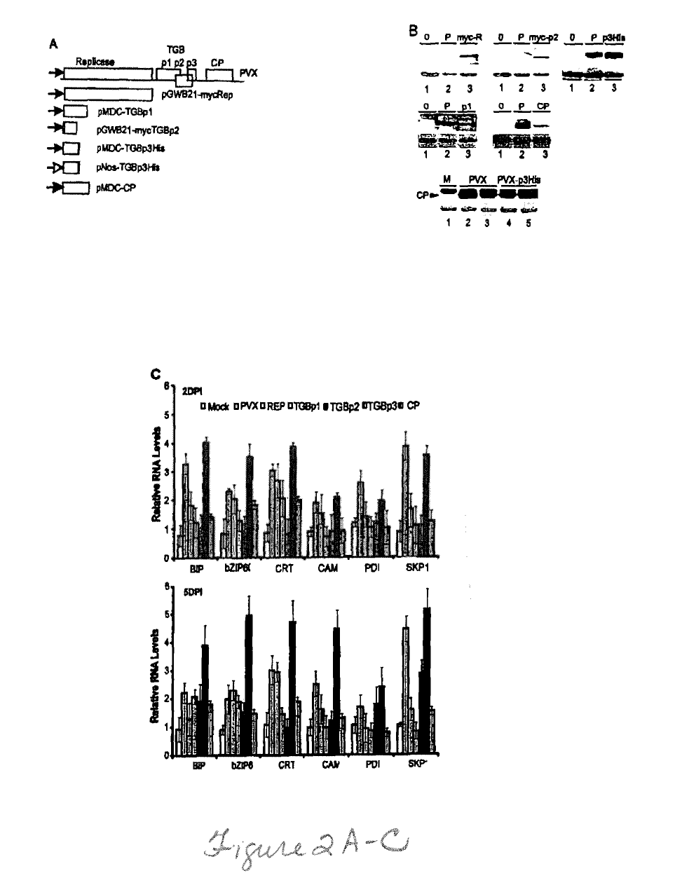

PVX infection leads to a general increase in population values (representing fold-changes in gene expression) for bZIP60 (FIG. 1) at 3 and 9 dpi. The median values at 9 dpi are 3 to 4-fold higher, the border of the box representing the upper 75^{th} percentile reaches 4 to 5-fold increase, and there is a maximum increase of 10-fold among outliers (FIG. 1; p<0.0001). Such a general increase allows us to conclude that the gene is induced.

We can also conclude that a gene is induced based on the box plot analysis whereby values have a positively skewed distribution. The median values for SKP1, BiP, PDI, CRT, and CAM at 0 dpi were approximately 0.9 with the range of values extending from 0.05 to 2.3 (FIG. 1). The median values for BiP and SKP1 increased to 3.4 fold at 9 dpi. The range of values for BiP and SKP1 expression are positively skewed (represented by elongation of box and whiskers above the median) and showed elevated values of 5.6- and 7.5-fold, respectively, and maximum values of 8- and 10-fold (FIG. 1; p<0.001).

PVX infection also leads to significant changes in CRT transcript accumulation, while CAM and PDI values show a mild positive change at 9 dpi. The boxes and whiskers for PDI, CRT, and CAM were generally small indicating low dispersion of values among the plants analyzed. For CAM, PDI, and CRT, the median values at 9 dpi increased to approximately 1.7, 2.0, and 2.5-fold respectively. CRT expression among plants in the 75^{th} percentile showed up to 4.5-fold increase (p=0.007) while for CAM and PDI the value ranges were moderately changed (FIG. 1; PDI, p<0.01; CAM, p=0.0143). The increased expression of bZIP60, BiP, CAM, PDI, and SKP1 clearly suggests that PVX infection coincides with the upregulation of UPR.

### TGBp3 Causes UPR-Related Gene Induction Following Agro-Delivery

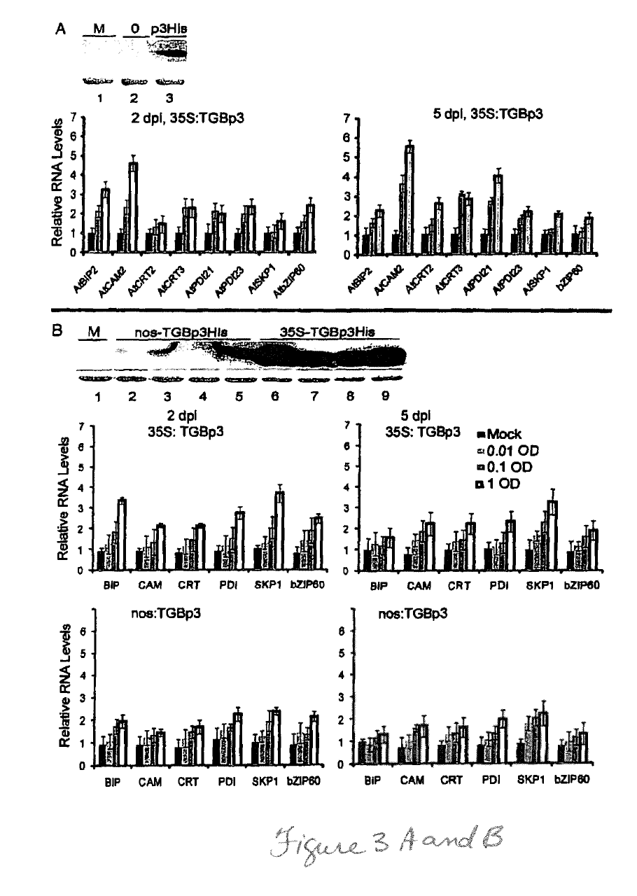

To study the mechanism of UPR induction and identify the viral inducers we employed Agrobacterium-mediated transient expression in a reproducible and quantitative assay. The entire PVX genome and each PVX gene was expressed from a binary vector containing the Cauliflower mosaic virus (CaMV) 35S promoter (FIG. 2A) for agro-delivery to N. benthamiana leaves. By comparing host gene expression following ago-delivery of each PVX gene we could learn whether UPR induction is a general response to virus infection or is specifically induced by a single PVX factor. PVX encodes three proteins that associate with the ER: replicase (Rep), TGBp2, and TGBp3 and any or all of these could cause upregulation of ER stress related genes. Given that UPR is often associated with ER stress, we predicted that one or more ER-resident factors would be responsible for host gene induction.

Initial immunoblot analysis was carried out to confirm PVX gene expression in N. benthamiana leaves following agro-delivery. Since we lack antisera detecting PVX replicase (Rep), TGBp2, or TGBp3 either a myc- or 6×-His tag was fused to these PVX genes (FIG. 2A) and immunoblot analysis was carried out using either anti-myc or penta-His antisera. We introduced the 6×-His tag into the PVX genome at the 3′ end of TGBp3 without altering virus infectivity but we were not able to make similar insertions to fuse myc tags to Rep or TGBp2. The 3′ end of Rep overlaps with the TGBp1 subgenomic promoter and adding a tag here would destroy the function of the subgenomic promoter. Also the TGBp2 promoter and coding sequence overlaps with TGBp1 and TGBp3 and a fusion in the endogenous sequence would eliminate the functions of the overlapping genes. However, the N-terminal myc fusion was expected to be functional, because GFP-TGBp2 fusions facilitated PVX infection (Ju et al., 2005). Thus immunoblot analysis in FIG. 2B confirms Rep and TGBp2 expression from the CaMV 35S promoter but cannot compare the levels of expression of the same genes from the PVX genome. On the other hand the levels of TGBp3, TGBp 1, and coat protein (CP) are comparable when expressed from the PVX genome or directly from the CaMV35S promoter (FIG. 2B).

N. benthamiana leaves were infiltrated with each binary construct, total RNA was extracted at 2 and 5 d post infiltration and qRT-PCR was carried out. Controls include leaves infiltrated with buffer (mock) or A. tumefaciens alone (Agro). Since agro-infiltration results in synchronous delivery of PVX and each PVX gene to plant cells, the values obtained were less dispersed than in plants inoculated with purified virus.

Interestingly, TGBp3-delivery resulted in 3.5- to 4-fold higher levels of BiP, bZIP60, CRT, and SKP1 transcript accumulation at 2 dpi in comparison to mock inoculated plants. At 5 dpi, BiP, bZIP60, CRT, CAM, and SKP1 showed 4- to 5-fold higher expression (FIG. 2C p<0.05). PDI induction was approximately 2-fold. Similarly the expression levels of bZIP60, BiP, CAM, PDI, and SKP1 in PVX-infected N. benthamiana leaves averaged 2 to 4-fold above the mock control at 2 d post infiltration (FIG. 2C; p≦0.1). Since the level of TGBp3 expression is comparable with the level of expression from the PVX genome, it is not likely that such high levels of bZIP60, BiP, CAM, PDI, and SKP1 are due to cytotoxic overexpression of TGBp3, but is more likely due to a real effect of TGBp3 on the host. Since the level of gene induction during PVX infection is not as profound as TGBp3 alone, it is reasonable to consider that there might be other viral proteins interacting with TGBp3 during virus infection which may suppress the effect of TGBp3 on the host.

We also notice 2-fold induction of BiP and bZIP60 by Rep and CP at either 2 or 5 dpi. TGBp2 also induces SKP1 although suggesting that its upregulation may be independent of bZIP60 controlled pathways. The effects of these other PVX proteins are not as profound as TGBp3. Notably CRT appears to be induced by PVX and several of its genes suggesting that its induction is more likely the result of a generalized response.

### UPR Induction in Arabidopsis

Given the microarray data identified the same set of host factors were induced in Arabidopsis and N. benthamiana plants, we employed the same TGBp3-containing binary vector to examine the ability of TGBp3 to induce UPR-related genes in Arabidopsis. Immunoblot analysis also confirmed successful expression of TGBp3His from the CaMV 35S promoter following agro-delivery to Arabidopsis leaves (FIG. 3A). For Arabidopsis, the average level of induction of AtbZIP60, AtBiP2 and AtCAM2 was 2 to 5.5-fold at 2 and 5 d post infiltration, indicating these are early and stable responses to the viral protein (FIG. 3A; p<0.05). AtCRT2 and AtPDI2-1 were upregulated at 5 d post infiltration (FIG. 3A; p<0.05). AtSKP1, AtCRT2, and AtPDI2 transcripts accumulated to significant levels ranging from 2 to 5.5-fold above control samples at 5 d post infiltration (FIG. 3A; p<0.05).

### Induction is Related to the Strength of the Promoter Driving TGBp3 Expression

We also examined whether gene induction in N. benthamiana is dependent upon the promoter driving expression or the concentration of A. tumefaciens infiltrated into the leaves. Leaves were infiltrated with A. tumefaciens carrying binary plasmids containing TGBp3 fused either to the NOS or CaMV 35S promoter. Protein expression was lower from the NOS promoter relative to the CaMV 35S promoter (FIG. 3B). Various dilutions of A. tumefaciens (OD₆₀₀=1.0, 0.1, 0.01) were delivered to N. benthamiana leaves to determine if there is a dosage dependent response. In general, host gene induction was greatest at 2 and 5 dpi when 1.0 OD₆₀₀ of A. tumefaciens solution was used and induction was proportionally less with each dilution (FIG. 3B). The NOS promoter is weaker than the CaMV35S promoter and this led to somewhat lower fold changes in expression of UPR-related genes (FIG. 3B). Collectively, these data demonstrate that expression of TGBp3 alone is sufficient to induce expression of UPR-related genes and that the TGBp3 levels correlate with the magnitude of induction.

Suppression of bZIP60 and its Impact on BIP and SKP1 Expression

BiP is an ER resident member of the Hsp70 family and its expression is a marker for ER stress and the UPR. bZIP60 is known to upregulate BiP as part of an ER stress response (Iwata and Koizumi, 2005) but it is not the only transcription factor responsible for its upregulation. SKP1 is a component of the SCF-type E3 ubiquitin ligase complex (Murai-Takebe et al., 2004) that is implicated in the elimination of misfolded proteins in mammalian and plant cells via the 26S proteasome (FIG. 9) (Wang et al., 2006) and it is unknown whether bZIP60 might also be responsible for its upregulation. Because bZIP60, BiP, and SKP1 expression was induced by TGBp3, we hypothesized that bZIP60 is an upstream transducer responsible for elevated levels of BiP and possibly SKP1. Importantly, FIG. 2 also shows that there is a 2-fold induction of BiP by other PVX factors and approximately 3-fold induction of SKP1 by TGBp2 at 5 dpi and therefore it is possible that both genes are only partially under the control of bZIP60. Therefore, silencing bZIP60 was expected to suppress BiP and SKP1 mRNA.

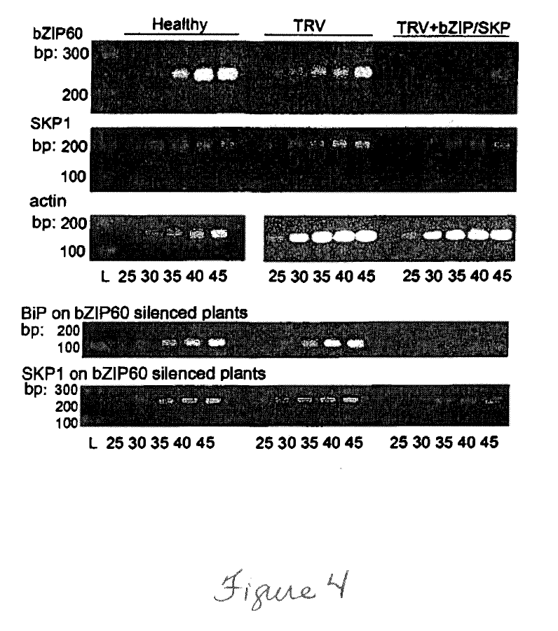

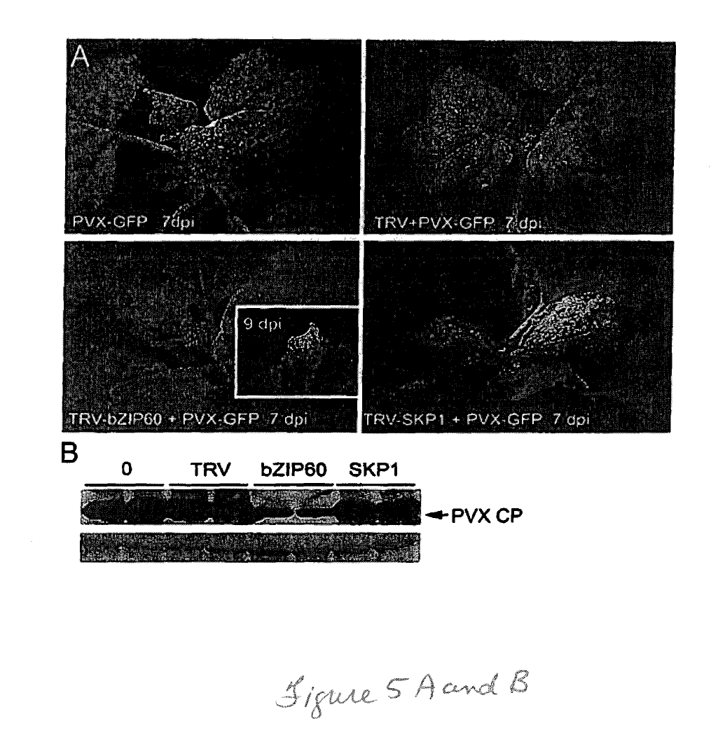

We employed Tobacco rattle virus (TRV)-based virus-induced gene silencing (VIGS) to knock down expression of bZIP60. A 600 by fragment of NbbZIP60 was cloned into the TRV vector (Ratcliff et al., 2001; Dong et al., 2007). N. benthamiana plants at the four leaf stage were pre-treated with buffer, TRV1 plus TRV2 empty vector, or TRV1 plus TRV2-bZIP60 or TRVI plus TRV2-SKP 1. RNA was harvested from upper leaves of silenced plants 14 days later, and then semi-quantitative RT-PCR was carried out to examine the expression of bZIP60, BiP, and SKP1 (FIG. 4). As expected, plants that were pre-treated with buffer or TRV alone showed similar levels of bZIP60, BiP, or SKP1. In plants treated with TRV2-bZIP60, bZIP60 was suppressed by 77% below mock treated plants. In plants treated with TRV2-SKP1, SKP1 levels were suppressed 60% below mock treated plants. We examined BIP and SKP1 expression in bZIP60 suppressed plants and found expression of these genes was also suppressed, 72% and 65%, respectively below mock treated plants. While we cannot assume that expression of these genes are solely driven bZIP60, these data shows that knocking down bZIP60 severely hampers BiP and SKP 1 expression. Thus, BiP and SKP 1 are downstream factors regulated by bZIP60.

Suppression of bZIP60 and SKP1 Reduces Local Infection and Systemic PVX Movement

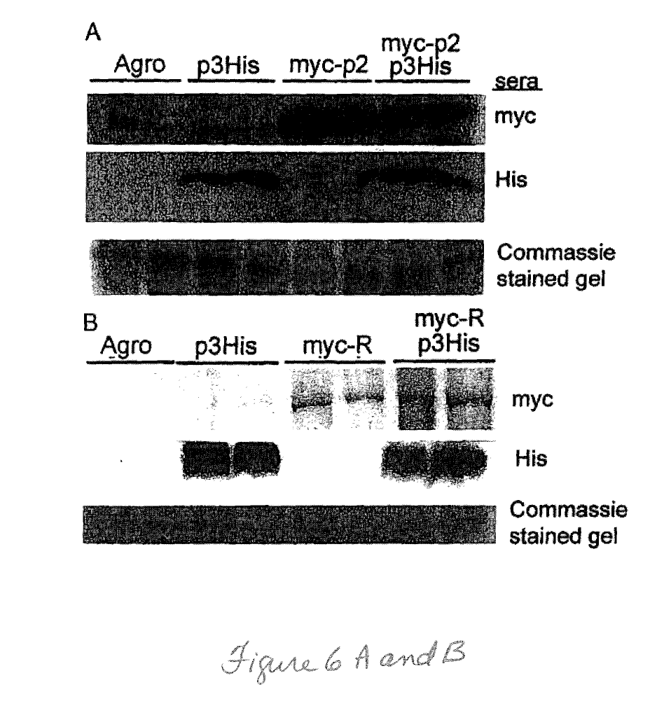

We inoculated plants with PVX-GFP and then monitored GFP fluorescence to determine if silencing bZIP60 interferes with the spread of virus infection throughout the plant. FIG. 9 shows that all plants that were pre-treated with buffer or TRV produced an average of 26-27 infection foci per leaf and became systemically infected with PVX-GFP by 5 dpi. bZIP60 silenced plants showed fewer infection foci (average of 18) and only 33% (4 of 12) became systemically infected with PVX-GFP at 5 dpi. We noted 75% of bZIP60 silenced plants became infected by 7 dpi, and 100% were infected by 9 dpi (Table 1; FIG. 5A). Thus, silencing bZIP60 slowed the spread of virus infection to the upper leaves. This conclusion is further supported by immunoblot analysis performed to detect PVX coat protein (CP) in systemically infected leaves at 7 dpi. PVX accumulation was greatly reduced in bZIP60-silenced plants in comparison to buffer or TRV pre-treated plants (FIG. 5B). These combined data indicate that bZIP60 is a contributing factor to optimum PVX accumulation in systemic tissues.

We also inoculated SKP1 silenced plants with PVX-GFP and then monitored GFP fluorescence to determine if SKP 1 is vital for the spread of virus infection. There were fewer infection foci (average of 19) on SKP1-silenced plants than on buffer or TRV pretreated leaves (Table 1). This is comparable to the numbers of infection foci occurring on bZIP60-silenced plants. At 5 dpi, 50% (6 of 12) of SKP1-silenced plants were systemically infected with PVX-GFP (FIG. 9). By 7 dpi, all SKP1-silenced plants were systemically infected with PVX-GFP. Thus, there is a slight delay in systemic infection compared to control plants (Table 1). Immunoblot analysis was performed to detect PVX coat protein (CP) at 7 dpi, in systemically infected leaves and there was no change in comparison to control plants (FIG. 5A).

With respect to SKP1 silenced plants, we observed higher GFP fluorescence in upper leaves although the immunoblot showed PVX accumulation in systemic tissues was unaltered. Given that SKP1 is a factor contributing to protein turnover, it is possible that silencing SKP1 reduced the turnover of GFP within infected cells. We have reported increased GFP accumulation in GFP-expressing transgenic leaves treated with a cocktail of proteasome inhibitors, which points to the likelihood that GFP can be a target for the 20S and/or 26S proteasome (Mekuria et al., 2008). We also reported a 5-fold reduction in the steady state levels of GFP in PVX-GFP infected protoplasts that were treated with tunicamycin, indicating that GFP turnover may be regulated by UPR machinery (Ju et al., 2008). Tunicamycin is often used as a chemical stimulus of the unfolded protein response (Leborgne-Castel et al., 1999; Surjit et al., 2007). Thus it is reasonable to consider that the greater intensity of fluorescence may not be an indicator of higher virus titer.

To determine if bZIP60 and SKP 1 contribute to virus accumulation in single cells, we delivered synthetic double strand RNAs (dsRNAs) targeting SKP 1 or bZIP60 for silencing (FIG. 5C). Protoplasts were harvested at 36 hpi and Northern analysis showed high levels of SKP1 and bZIP60 in untreated BY-2 protoplasts, but barely detectable levels of the same transcripts in protoplasts treated with dsRNAs. These data indicate that the dsRNAs can successfully knock down host gene expression. PVX-GFP transcripts were delivered to untreated and dsRNA treated protoplasts and northern analysis was carried out at 36 hpi. PVX genomic RNA accumulation was unaffected by silencing SKP1 but was significantly impeded in bZIP60 silenced protoplasts. These data indicate that bZIP60, but not SKP1, is a factor in virus replication

Since we showed in FIG. 4 that bZIP60 might regulate expression of SKP1, but knocking down each gene has a different outcome in isolated protoplasts it is arguable that SKP1 does not play the same roll in virus replication as bZIP60. bZIP60 may regulate other genes that directly affect PVX replication, in addition to contributing to the regulation of SKP1 expression. In plants, knocking down bZIP60 seem to contribute to reduce or delay virus systemic accumulation, although knocking down these genes have different affects on GFP intensity in systemic leaves (FIG. 5, Table 1). While the data thus far argues that this gene network contributes to PVX infection, based on these experiments it is reasonable to conclude that bZIP60 regulates more than one gene that contributes to optimum PVX infection.

TABLE 1
Local and systemic PVX infection on silenced plants

| | Average No. of | No. Infected | No. Infected |
|---|---|---|---|
| Treatment | Foci per Leaf^{a} | at 5 dpi^{b} | at 7 dpi^{b} |
| Buffer + PVX-GFP | 26 +/− 9 | 8/8 | 8/8 |
| TRV + PVX-GFP | 27 +/− 11 | 8/8 | 8/8 |
| TRV-bZIP60 + PVX-GFP | 18 +/− 10 | 4/12 | 9/12^{c} |
| TRV-SKP1 + PVX-GFP | 19 +/− 8 | 6/12 | 12/12 |

^{a}The average number of green fluorescent infection foci was determined using 16 to 19 leaves at 5 dpi.
^{b}Total number of systemically infected plants relative to the total number of plants that were inoculated. Plants were scored based on the presence of systemic disease and green fluorescence in the upper leaves.
^{c}All bZIP60-silenced plants were systemic at 9 dpi.

### TGBp3 Expression Does Not Significantly Influence the Turnover of TGB2 or Replicase

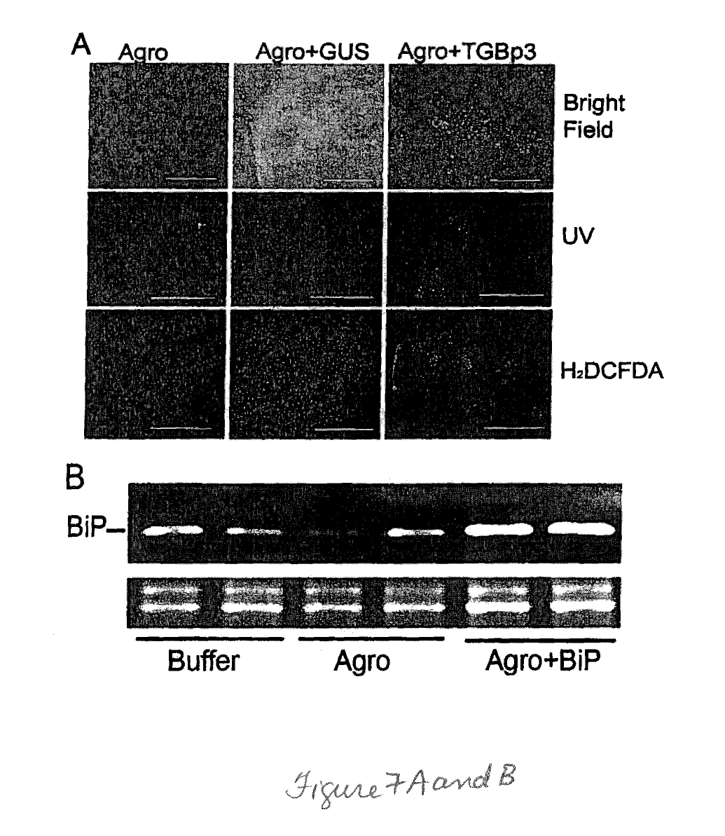

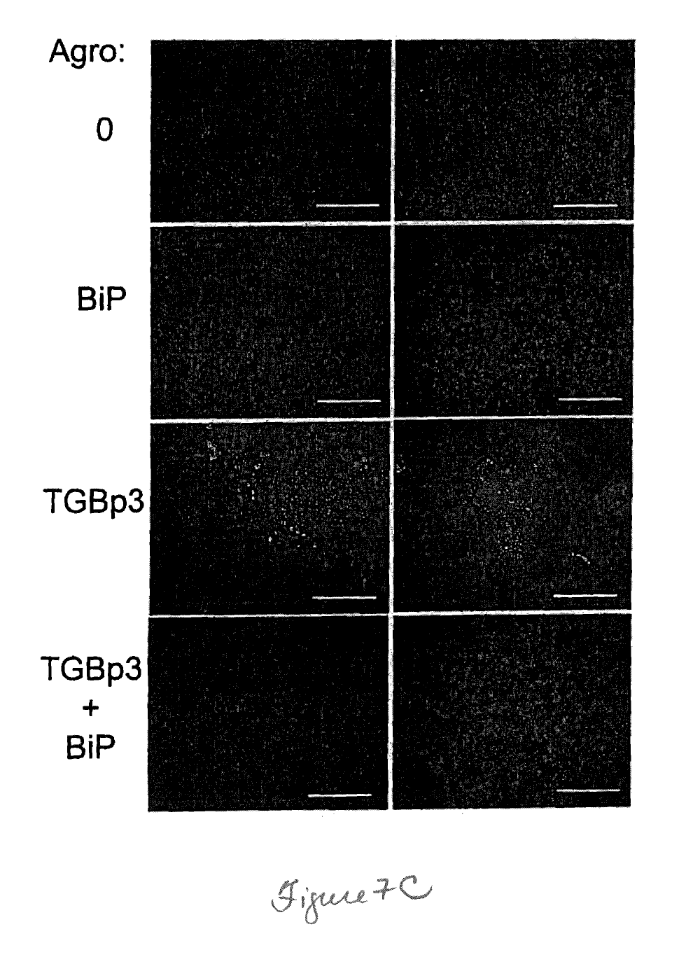

Many mammalian viruses regulate virus replication via UPR. They encode proteins that insert into the ER membrane and stimulate UPR which targets the viral replicase for degradation as a means to downregulate virus replication late in infection (Yu et al., 2006; Medigeshi et al., 2007). Since PVX replicase, TGBp2, and TGBp3 associate with the ER we considered the possibility that TGBp3 stimulates UPR as a means to regulate turnover of other PVX proteins in the ER. Also, given the differences in GFP intensity when expressed from the PVX genome in SKP1-silenced plants, it seemed reasonable to consider that TGBp3 might stimulate cellular UPR to regulate accumulation of other PVX proteins. To examine this hypothesis, TGBp3His and mycTGBp2 or TGBp3His and mycRep (replicase) were co-expressed in N. benthamiana leaves using agro-infiltration and immunoblot analysis was used to detect the epitope-tagged proteins at 3 d post infiltration (FIG. 6). Leaves were also infiltrated with A. tumefaciens as controls. Consistently high levels of TGBp3-His were detected when it was expressed alone or co-delivered with mycTGBp2 or mycRep. TGBp3 did not appear to have a significant impact on the accumulation of mycTGBp2 or mycRep.

Ectopic Expression of TGBp3 can Lead to Cell Death which can be Alleviated by BiP Overexpression.

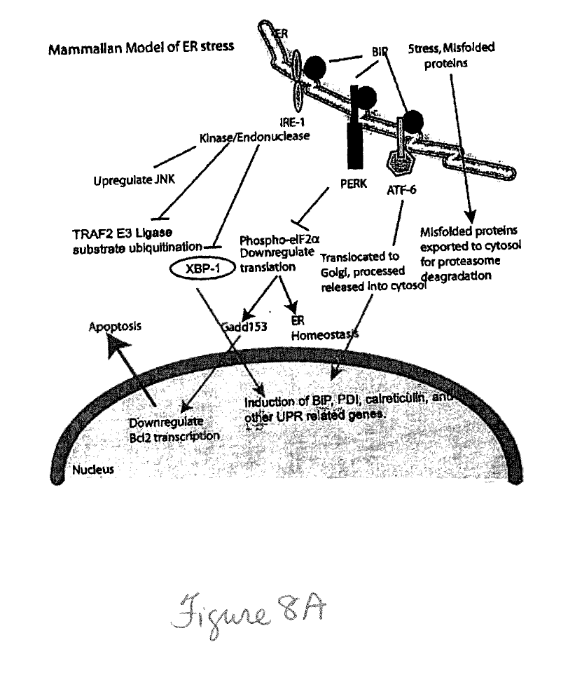

We re-examined leaves that were agro-infiltrated with plasmids expressing TGBp3 from the CaMV 35S promoter and noted microscopic necrotic lesions (FIG. 7A) which are absent from control agro-delivery of β-glucuronidase (GUS) coding sequences or A. tumefaciens alone. HR necrosis is evident from blue autofluorescence seen under UV light and ROS activity was detected using the fluorogenic probe 2′,7′-dichloro-fluorescein diacetate (H₂DCFDA) (FIG. 7A). To determine whether BiP is directly responsible for TGBp3-related HR or represents a pathway branch that is induced by TGBp3 alongside the cell death signaling pathway, we over expressed the NbBLP-4 (BiP) coding sequence from the pBI121 plasmid. N. benthamiana leaves were infiltrated with A. tumefaciens containing pBI-NbBLP-4, pBI121 alone, or with buffer and immunoblot analysis was used to compare BiP protein levels among N. benthamiana leaves harvested 2 d after infiltration (FIG. 7B). The density of bands reporting BiP expression were 3-fold higher in NbBLP-4 infiltrated leaves than buffer treated leaves (data not shown). This level of overexpression is within the range reported at 3 dpi for PVX-infected leaves and agro-infiltrated leaves expressing TGBp3.

We agro-infiltrated leaves delivering BiP alone (pBI-NbBLP-4), TGBp3 alone, or a mixture of Agrobacterium expressing BiP and TGBp3 (FIG. 7C). BiP overexpression was sufficient to alleviate TGBp3 induced necrosis (FIG. 7C). Using a UV lamp and H₂DCFDA staining, necrosis was seen only in TGBp3 expressing leaves. Co-delivery of BiP eliminated necrosis and evidence of ROS (FIG. 7C). These data concur with earlier findings that BiP is upregulated upon pathogen invasion as a response to the increase in protein translation, but is not directly responsible for HR (Jelitto-Van Dooren et al., 1999). Importantly, the fact that necrosis is abrogated by BiP overexpression clearly demonstrates that TGBp3-related cell death is linked to ER stress. Given that FIG. 4 shows bZIP60 is a factor regulating BiP expression as well as SKP1, these data argue that bZIP60 and BiP might play a role in regulating cytotoxic effects of PVX proteins during virus infection. Limiting protein cytotoxicity might be important for enabling optimal systemic virus spread by reducing tissue necrosis.

### Results

Here we report that bZIP60, several plant UPR related ER resident chaperones, and the Cullin co-chaperone SKP1 are induced following PVX infection or A. tumefaciens delivery of TGBp3 to N. benthamiana or Arabidopsis plants. Evidence that PVX infection, and ectopically expressed TGBp3 upregulates bZIP60, SKP1, and ER resident chaperones such as BiP is intriguing and provides the first clear evidence that a plant viral protein elicits UPR and a factor (SKP1) linked to proteasome-dependent pathways. Such comparisons of Arabidopsis and N. benthamiana gene expression in response to TGBp3 delivery are significant because they demonstrate that this is not a host-specific response and that there are general implications for host-virus interactions. Unfortunately, the incomplete representation of host genes on the microarrays and the lack of the complete N. benthamiana genome sequence hinder identification of orthologous genes or explain why certain genes such as bZIP factors, were not identified in Arabidopsis although they were identified in the potato microarrays. However, in a broader context, the data shows that members of gene families encoding ER resident proteins can be induced to similar levels (between 2 and 5.5 fold) in both species.

bZIP60 belongs to a class of membrane bound ER stress sensors that is responsible for up regulating genes involved in UPR (FIG. 8) (Iwata and Koizumi, 2005; Urade, 2007; Lu and Christopher, 2008). bZIP60 is activated by ER stress and regulated by intramembrane proteolysis. Cleavage of the full-length protein by a non-canonical proteolytic event releases the transcription factor from the ER (Iwata et al., 2008; Iwata et al., 2009). The truncated bZIP60 activates promoters containing cis elements, P-UPRE and ERSE, which are responsible for ER stress response, including activating its own transcription and BiP genes (Seo et al., 2008; Urade, 2009) (FIG. 1). bZIP60 regulated UPR is potentially a factor in promoting optimal PVX accumulation in infected protoplasts and plants by: a) reducing cytotoxicity that can lead to necrosis and; b) regulating expression of cellular factors contributing to virus infection. These conclusions are based on critical observations. First, our observations that agro-infiltration of NbBLP-4 eliminated TGBp3-induced HR confirmed a cytoprotective role for BiP in virus-infected leaves and for controlling TGBp3-induced ER stress (FIG. 1) (Iwata et al., 2008; Lu and Christopher, 2008; Urade, 2009). These experiments suggest that PVX employs the UPR machinery, via TGBp3 and BiP, to regulate cytotoxic damage to the cell as a means to promote virus spread. UPR is reported to be a component of important early responses to pathogen invasion in anticipation of the increase in protein synthesis along the ER, but is not directly responsible for defense gene induction (Jelitto-Van Dooren et al., 1999). In particular, BiP is a well known component of cellular cytoprotective responses to alterations in the ER or accumulation of misfolded proteins and controls the status of certain UPR transmembrane signal transducers (e.g. IRE1, PERK, and ATF6) (Tardif et al., 2004; Zhang and Kaufman, 2006). Lebourne-Castel et al. (1999) were the first to demonstrate that mild overexpression of BiP (NbBLP-4) in transgenic plants restores ER homeostasis and protects plants from ER stress (Leborgne-Castel et al., 1999; Costa et al., 2008). Importantly, AtHSP70 induction is also linked to plant protein overexpression. BiP is a member of the HSP70 multigene family, and a subset of cytoplasmic AtHSP70 genes (HSC70-1, -2, -3, and HSP70 but not AtHSP70B) are induced as part of a general response to viral protein accumulation (Whitham et al., 2003; Aparicio et al., 2005). Thus HSP70 may contribute to modulating cellular stresses during virus infection in a manner that is reminiscent of the UPR (Aparicio et al., 2005). Our study contrasts with the prior work on HSP70 by presenting several experimental outcomes which point to TGBp3 as a specific inducer of BiP expression.

To support the notion that UPR is a factor regulating disease, we inoculated bZIP60 and SKP1-silenced plants and protoplasts with PVX-GFP. We reported fewer infection foci on bZIP60 and SKP1-silenced N. benthamiana plants, suggesting that PVX infection was attenuated by the greatly reduced bZIP60 or SKP1 expression. The reduced number of green fluorescent infection foci (Table 1) and reduced PVX genomic RNA accumulation in protoplasts correlated with the dramatic reduction in bZIP60 transcript accumulation. These data suggest that bZIP60 is a factor contributing to PVX-GFP replication in protoplasts and N. benthamiana leaves. The partial inhibition of bZIP60 expression in N. benthamiana plants did not compare to the inhibition seen in protoplasts. Therefore new research tools, are needed improve knockdown of bZIP60 in N. benthamiana plants or other hosts of PVX to examine the contribution of bZIP60 to promoting long distance PVX spread. It is worth considering bZIP60 as a target for developing a transgenic approach to virus resistance.

SKP1 is an essential component of the SCF family of E3 ubiquitin ligases which provides substrate ubiquitination preceding proteasome-mediated degradation (Cardozo and Pagano, 2004; Petroski and Deshaies, 2005). Typical substrates are cellular proteins crucial for eukaryotic physiology and defense. In FIG. 2, we report that PVX infection and TGBp3 upregulates SKP1 mRNA expression, but it is not clear if increased SKP1 expression is necessary to degrade viral proteins or simply to alleviate congestion of proteins in the ER by enhancing protein turnover.

We also noticed that SKP1 is upregulated by TGBp2 at 5 dpi although TGBp2 does not appear to be impacted bZIP60 expression. We also showed that silencing bZIP60 can alter expression of SKP1 in the absence of a viral inducer which suggests that these genes are linked in a pathway. But failure to completely shut down SKP1 expression, combined with evidence that TGBp2 can induce SKP1 raises the possibility that SKP1 may be controlled by additional factors and may not be completely controlled by bZIP60.

Evidence linking TGBp3 to SKP 1 is exciting given recent reports linking proteasomal activities to systemic virus movement and to the function of certain of plant viral silencing suppressor proteins. For example, RPN9 is a proteasomal subunit whose expression is required for systemic movement of Tobacco mosaic virus and Turnip mosaic virus. Silencing RPN9 seemed to generally impede virus systemic movement, although the protein also seems to play a role in appropriate vascular development (Jin et al., 2006). More closely related to this work is evidence that the Beet western yellows virus (BWYV) P0 silencing suppressor has an F-box domain and directly binds SKP1 orthologs in Arabidopsis, although the cellular components of the silencing machinery that are targeted by the SCF E3 ubiquitin ligase are not known (Baumberger et al., 2007). However, silencing SKP1 in N. benthamiana caused plants to become resistant to BWYV infection indicating the relationship of P0 and SKP1 is vital for virus spread. We report here that silencing SKP1 reduced the number of infection sites on inoculated leaves and the number of infected plants (Table 1) suggesting that it plays a role in PVX infection. Moreover, the PVX TGBp1 silencing suppressor targets AGO1, which is the effector nuclease of RNA silencing machinery, for degradation via the proteasome (Chiu et al., 2010). Thus combining this work with previous reports of TGBp1, it is possible that TGBp3 upregulates UPR and components of the ubiquitin-proteasome pathway to enable degradation of AGO1 mediated by TGBp 1. Thus the TGBp 1 and TGBp3 proteins might act in concert to regulate host defense and stress responses in a manner that renders plants more susceptible to PVX infection.

bZIP60 could play a direct role in PVX infection that is unrelated to its role in UPR induction. This explanation seems unlikely given that bZIP60 is responsible for up regulation of ER resident chaperones such as BiP (Iwata and Koizumi, 2005; Lu and Christopher, 2008), and we show that BiP plays a role in suppressing TGBp3-related ER stress. Second, bZIP60 might upregulate another gene whose protein product is a positive factor in promoting virus replication and movement. Further experiments are needed to identify additional bZIP60-regulated factors and assess their role in PVX movement. Third, bZIP60 might be required to enhance cellular protein folding abilities (perhaps by increasing BiP expression), proteasomal function for degradation of AGO1, and ER membrane synthesis necessary for optimal virus accumulation. This latter possibility is based on the flavivirus model. The nonstructural proteins of JEV and DEN-2 trigger the ER resident sensors which lead to signaling pathways that enhance cellular protein folding abilities, ER membrane synthesis, and up regulation of the secretory system (Urano et al., 2000). These events are necessary to manage the increase in protein translation resulting from virus infection and provides further membranes needed for replication and maturation (Yu et al., 2006). During PVX infection, expansion of the ER network is known to be important for virus infection. Cells treated with cerulenin, an inhibitor of membrane synthesis, supported reduced virus replication (Bamunusinghe et al., 2009). Thus the preliminary data point to the possibility that PVX, similar to flaviviruses, triggers UPR to enhanced cellular protein folding abilities and ER membrane synthesis. If SKP1 expression is regulated by bZIP60 then there is additional regulation of the proteasomal pathway that might be important for effective silencing suppression mediated by TGBp1. These combined events could be necessary to promote virus cell-to-cell movement.

Similarly, build-up of viral proteins in the ER or the retention of inefficiently folded viral envelope proteins in the ER is cytotoxic and leads to UPR initiation by mammalian viruses such as HCV, JEV, human cytomegalovirus, and bornavirus (Chan and Egan, 2005; Williams and Lipkin, 2006). In these examples, viral proteins trigger ER stress in a manner that leads to cell death only when the protein load in the ER exceeds the folding capacity induced by ER stress. When we compare the effects of expressing TGBp3 at various levels, it becomes worth considering that the amounts of TGBp3 expressed during PVX infection are tightly regulated to avoid damaging the cells. Given that TGBp3 is expressed from the PVX genome via a subgenomic RNA at low levels, this may be necessary to promote virus spread by preventing cytotoxic cell death.

There are intriguing similarities between PVX TGBp3 and the HIV Vpu protein. Both proteins are expressed from bicistronic mRNAs, and they have low molecular weights with single transmembrane domains that insert into the ER. Vpu binds to the cellular CD4 protein in the ER and recruits the human F-box protein βTrCP targeting CD4 for degradation via the ubiquitin-proteasome pathway. CD4 is a cell surface receptor required for HIV uptake into cells and the process of dislocation and degradation of CD4 in the ER reduces the number of available receptors at the cell surface and is important to free HIV gp160 in the ER for virus maturation and trafficking (Bour et al., 1995; Schubert et al., 1998; Malim and Emerman, 2008; Nomaguchi et al., 2008). It is worth considering that TGBp3 might function to bind cellular proteins and recruit them to the ubiquitin-proteasome pathway. PVX TGBp3 might function in the ER to down regulate host factors contributing to virus replication or early stages of infection during their translation which could be essential for maintaining virus infection and promoting cell-to-cell spread.

### Conclusion

For the last 15 years plant virologists have reported viral movement proteins embedded in the ER. Until now researchers have viewed the ER as a location for assembly and lateral transport of movement complexes toward plasmodesmata, but there have been no reports indicating a role for the ER or UPR in promoting plant virus spread. This is in contrast to significant advances on this topic that have been made in mammalian virus research. The data presented in this study point to a new role for the ER in regulating plant virus movement. We provide the first evidence linking UPR to systemic accumulation of PVX and show that bZIP60 is an important factor in PVX infection.

### Materials and Methods.

### Plasmids and Bacteria Strains.

pGR208 is a binary vector containing PVX-GFP genome was obtained from Dr. P. Moffett (University of Sherbrooke). pGR208 is deliverable to plants by agro-infiltration. A 6×-His tag (underlined) was introduced at the 3′ end of the PVX TGBp3 coding sequence in pTXS-GFP plasmids using the Quick-change II XL Site-Directed Mutagenesis Kit (Stratagene, La Jolla, Calif.), forward primers (5′-GTTGACGGTTAAGTTTCACCATCACC ATCACCATTGATACTCGAAAG-3′, SEQ ID NO: 1) and reverse primers (5′-CTTTCGAGTATCAATGGTGATGGTGATGGTGAAACTTAACCGTTCAAC-3′, SEQ ID NO: 2). The reaction products were transformed into E. coli XL10-Gold.

A. tumefaciens deliverable binary vectors were prepared using pMDC32 plasmids and Gateway Technology with Clonase II (Invitrogen, Carlsbad, Calif.). The pMDC32 contains the CaMV 35S promoter. To generate pMD32-TGB3His, the TGB3His DNA fragment was first amplified using with attB1 primers (GGGGACAAGTTTGTACAAAAAACAGGCTTCGG ATCCATGGAAGTAAATACATATC, SEQ ID NO: 3) and attB2 primers containing 6×-His tag (underlined), (5-GGGGACCACTTTGTACAAGAAAGCTGGGTCTCAGTGGTGATGGTGA TGATG GAAACTTAACCGTTCAAC, SEQ ID NO: 4). To generate pMD32-TGBp1 and CP, PCR fragments were amplified using attB1 primers (GGGGACAAGTTTGTACAAAAAA GCAGGCTGTATGGATATTCTCATCAGTAG, SEQ ID NO: 5; GGGGACAAGTTTGTAC AAAAAAGCAGGCTGTATGTCAGCACCAGCTAGC, SEQ ID NO: 6) and aatB2 primers (GGGGACCACTTTGTACAAGAAAGCTGGGTGCTATGGCCCTGCGCGGACAT, SEQ ID NO: 7; GGGGACCACTTTGTACAAGAAAGCT GGGTGTTATGGTGGTGGTAGAGTGAC, SEQ ID NO: 8). The DNA fragments were incubated with pDONR/zeo and BP clonase II for one h. Then pMDC32 binary vector (obtained from Dr. R. Sunkar, Oklahoma State University) and LR clonase II were added to the reaction mix and incubated for one h. The reaction products were transformed to One Shot OmniMax2-T1 competent E. coli cells. After sequencing confirmation of the derived plasmids, the pMDC32-TGBp3his plasmid was used to transform to A. tumefaciens strain GV2260. A set of plasmids were also prepared replacing the CaMV35S promoter with the NOS promoter. 307 nucleotides of NOS promoter is amplified from pBI121 plasmid using the forward primer: GCAAGCTTGATCATGAGCGGAGAATTAAG, SEQ ID NO: 9 (Hind III site is underlined) and a reverse primer: GCGGTACCAGATCCGG TGCAGATTATTTGG, SEQ ID NO: 10 (Kpn I site is underlined). The PCR fragment was cloned into pMDC-p3H between Hind III and Kpn Ito replace its CaMV 35S promoter.

The A. tumefaciens deliverable pGWB21 binary vector (obtained from Dr. T. Nakagawa, Shimane University, Japan) which includes a 11×myc tag at the 5′ end of inserted open reading frames. PVX TGBp2 and replicase coding sequences were cloned into pGWB21 using the same Gateway Technology described above. To generate pGWB21-mycTGB2, TGB2 was PCR amplified using forward attB1 primers containing the myc tag (underlined) (5′-GGGGACAA GTTTGTACAAAAAAGCAGGCTTCGGATCCATGGAACAAAAAATTTCTAAGATCTGTCCGCGCAGGGCCATAGG-3′, SEQ ID NO: 11) and reverse attB2 primers (5′-GGGGACCA CTTTGTACAAGAAAGCTGGGTACTACTAATGACTGCTATGATTGTTACC-3, SEQ ID NO: 12′). To generate pGWB21-mycRep, the PVX replicase was PCR amplified using forward attB1 primers containing the myc (underlined) tag (5′-GGGGACAAGTTTGTACAAA AAAGCAGGCTTGATGAACAGAAACTTATTTCTGGAAGAAGATCTGGCCAAGGTGCG CGAGGTT-3′, SEQ ID NO: 13) and reverse attB2 primers (5′-GGGGACCACTTTGTACAA GAAAGCTGGGTCTTAAAGAAAGTTTCTGAGGCG-3′, SEQ ID NO: 14). After sequencing confirmation, pGWB21-mycTGBp2 and pGWB21-mycRep were transformed to A. tumefaciens strain GV2260.

A. tumefaciens LBA4404 containing pBI-BLP4 was prepared by inserting the coding sequence between XbaI and Sad restriction sites of pBI121 plasmids (Jefferson et al., 1987). Total RNA extracted from N. benthamiana leaves using Trizol Reagent (Invitrogen, Carlsbad, Calif.) and treated with DNase I (Promega, Madison, Wis.). NbBLP-4 coding sequence (Accession No FJ463755) was synthesized with Superscript Reverse Transcriptase III (Invitrogen) and amplified by Pfu Turbo DNA polymerase (Stratagene). NbBLP-4 cloning primers are designed based on NtBLP-4 (Accession No. X60057) (FIG. 9). The NbBLP-4 showed greater than 98% homology with the NtBLP-4 cDNA (Leborgne-Castel et al., 1999). NbBLP4 cDNA was cloned into pGEM-T Easy vector (Promega) and sequenced with M13 primers. The plasmid pRTL2.TGBp3-GFP was prepared previously (Samuels et al., 2007). TGBp3Dm1-GFP was PCR amplified using primers containing NcoI and BamHI restriction sites and then inserted into pRTL2 plasmids.

A. tumefaciens strain GV2260 containing TRV1 plus TRV2-NbSKP1, TRV2-bZIP60, TRV2-PDS or TRV2-GFP was kindly from Dr. S. Dinesh-Kumar (Yale University) (Liu et al., 2004; Bhattarai et al., 2007). NtbZIP60 gene fragment (168-776 nt of NtbZIP60 coding sequence) was cloned into pDONR/zeo and pTRV2 gateway vectors with Gateway Technology Clonase II (Invitrogen). forward primer attB1 (5′-GGGGACAAGTTTGTACAAAAAAGCAGG CTGTCGGTCACGTGGCTGTC-3′, SEQ ID NO: 15, and reverse primer attB2 (5′-GGGGACC ACTTTGTACAAGAAAGCTGGGT GGAACCAGAAGACCGTGG-3′, SEQ ID NO: 16).

### Plant Materials and Inoculations.

N. benthamiana and Arabidopsis plants were used. Purified virus was prepared from infected N. benthamiana plants using the traditional methods, and suspended in 0.01M phosphate buffer (pH7.0) (Shadwick and Doran, 2007). Virus concentration (C) was determined by measuring OD₂₆₀, and calculated by the formula C=OD₂₆₀/3.0. Aliquots of viruses are stored in −80□ and then 30 μg/ml virus was used for each inoculation.

Agro-infiltration for plasmid or TRV delivery to N. benthamiana or Arabidopsis leaves was performed with 1 ml needle-free syringe according to published protocols (Liu et al., 2002). A. tumefaciens LBA4404 or GV2260 infiltrations were conducted using ten plants for each treatment and infiltration medium (buffer) was used as negative control. A. tumefaciens cultures were collected by centrifugation, and resuspended in agro-infiltration solution (10 mM MgCl₂, 10 mM MES, pH 7.0, 204 μM acetosyringone). The suspension was adjusted to OD₆₀₀=1.0, 0.1, or 0.01 and infiltrated to N. benthamiana leaves with 1 ml needle-free syringe. N. benthamiana plants were grown to a four-leaf stage for infiltration. GV2260 cells were also infiltrated to leaves serving as the negative control.

### H₂DCFDA Staining of Leaf Segments.

N. benthamiana were agro infiltrated and ROS activity was detected using the fluorogenic probe H₂DCFDA (Mahalingam et al., 2006). For visual assessment of ROS activity, leaf samples were treated with 50 μM H₂DCFDA for 20 min and observed using a Nikon E600 epifluorescence microscope.

### Immunoblot Analyses.

For immunoblot analysis of virus infected or agro-infiltrated leaves, 0.3 g treated leaf samples were harvested from inoculated leaves at 5 dpi or upper leaves at 8-10 dpi. Total protein was extracted from leaves by grinding samples with extraction buffer (4M urea, 4% SDS, 0.2M DTT, 20% glycerol, 0.2M Tris-HCl, pH6.8, and 0.04% bromophenol blue) (Draghici and Varrelmann, 2009) or standard protein extraction buffer (100 mM Tris-HCl, pH7.5, 10 mM KCl, 0.4M sucrose, 10% glycerol, 10 μM PMSF) (Sambrook, 1989) and quantified using Bradford Reagent (Sigma-Aldrich, St. Louis, Mo.). Thirty μg, protein for each sample was load onto 4-20% precast gradient or 10% SDS-PAGE (Bio-Rad), electroblotted to Hybond-P (GE Healthcare, Piscataway, N.J.).) using standard protocols (Sambrook, 1989). Blots were probed with BiP (GRP78) antiserum (Affinity BioReagents, Golden, Colo.), 5×-His monoclonal IgG (Qiagen, Valencia, Calif.), c-Myc monoclonal IgG (Santa Cruz Biotechnology, Santa Cruz, Calif.), PVX coat protein (Agdia, Elkhart, Ind.) or GFP polyclonal antiserum (Affinity BioReagents). HRP conjugated goat anti-mouse IgG (Jackson ImmunoResearch Laboratories, West Grove, Pa.) served as the secondary antiserum using ECL Advanced Western Blotting Kit (GE Healthcare). Blots were exposed to film for 10-60 seconds. Film was scanned using Alpha Image imaging system (Alpha Innotech. San Leandro, Calif.) and the reverse image was recorded. Densitometric analysis was performed by Alpha Ease FC software (Alpha Innotech). Films are scanned and images are cropped with scanner CanonScan 9950F and associated program Arcsoft Photo Studio 5 (Canon USA). qRT-PCR Analysis of Infected Leaves and Semi-quantitative RT-PCR of Silenced Plants.

Mock inoculated (treated with agro-infiltration buffer) was used as a control and calibrator sample. SV Total RNA Isolation kit (Promega Corp.) was used to extract total RNA from samples. The first strand cDNA was synthesized by Superscript reverse transcriptase III (Invitrogen) using hexamer random primers. qPCR was carried out using 25 □l reactions and 100 to 900 nM primers designed using the coding sequences for known Arabidopsis, N. tabacum or N. benthamiana genes (FIG. 9). Initial RT-PCR tests of the gene specific primers confirmed their ability to amplify single bands of the predicted sizes from N. benthamiana cDNA. 25 ng cDNA was used to perform qPCR using the Power SYBR Green II Master Mix and ABI 7500 PCR machine (Applied Biosystem, Foster City, Calif.). Reactions were incubated first at 95° C. for 10 min, and then 40 cycles of 95° C. for 15 sec and 60° C. for 60 sec. Efficiencies of all primers were verified by normal RT-PCR and gel electrophoresis. The comparative C_{T} method was employed for relative quantitation of gene expression following virus treatments. qRT-PCR efficiencies were determined by control amplifications using 0.01, 0.1, 1, 10, 100 ng of template cDNA. Duplicate PCR reactions for each sample were carried out and averaged. The comparative C_{T} method employs the formula 2^{-dd CT} where the values of the endogenous control (18S RNA) and calibrator (constant quantity of healthy sample template) and are subtracted from the target sample value to provide the ddC_{T} value. The 2^{-dd CT} represents the fold of RNA accumulation.

Semi-quantitative RT-PCR NbSKP1 and bZIP60 primers were designed to anneal outside the target sequence of virus-induced gene silencing (Liu et al., 2002). To detect SKP1 or bZIP60 silencing effect, total RNA was extracted from agro-infiltrated N. benthamiana leaves at 14 days post-infiltration with SV Total RNA Isolation System (Promega). The first strand cDNA was synthesized with SuperScript III Reverse Transcriptase (Invitrogen), 1 μg total RNA and 100 μM 6-mer random primers. bZIP60 semi-quantitative RT-PCR was performed with bZIP60 primers forward 5′-CCTGCTTTGGTTCATGGGCATCAT-3′, SEQ ID NO: 17 (672-695 nt), reverse 5′-CACATCACAATTCCCAAATAATG-3′, SEQ ID NO: 18 (877-900 nt). For amplification of NbSKP1 we employed forward primers (5″-TGACATGCCAGACAGTT GCAGACA-3′, SEQ ID NO: 19) (303-326 nt), and reverse primers (5′-TTCGAT CTCATCCTGGCT-3′, SEQ ID NO: 20) (439-462 nt). N. benthamiana actin primers forward (5′-AAAGACCAGCTCATCCGTGGAGAA-3′, SEQ ID NO: 21), and reverse primer (5′-TGTGGTTTCATGAATGCCAGCAGC-3′, SEQ ID NO: 22) were used to amplify actin as the internal control. Semi-quantitative RT-PCR was performed with same protocols described above.

Preparation of BY-2 Protoplasts, dsRNA Delivery, and Northern Analysis

BY-2 protoplasts are prepared and transfected as previously described (Lee et al., 2008). Two μg of NbSKP1 or NtbZIP60 dsRNAs, and 25 μg PVX-GFP transcripts were delivered to 1×10⁶ protoplasts. Transfected BY-2 cells were incubated at 25° C. and then total RNA was extracted at 48 h using TRIzol Reagent (Invitrogen). 15 μg total RNA was subject to Northern detection with the North-2-South Chemiluminescent Hybridization and Detection kit (Pierce Biotechnology, Rockford, Ill.).

PVX-GFP transcripts were prepared using standardized protocols that were previously reported (Bamunusinghe et al., 2009). dsRNAs were prepared as previously described (Silva et al., 2010) (Qi et al., 2004). A PCR fragment of NbSKP1 coding sequence (21-457 nt) and a fragment of NtbZIP60 coding sequence (168-776 nt) were cloned into pGEM-T Easy (Promega). The plasmids were linearized using Spe I and positive sense transcripts were synthesized using RiboMAX Large Scale RNA Production System-T7 (Promega). Plasmids were also linearized using Nco I and then negative sense transcripts were synthesized using RiboMAX Large Scale RNA Production System-SP6 (Promega). DNA was removed by digestion for 15 min using DNAse I. Transcripts were precipitated and then resuspended in RNAse-free ddH₂O. Equal amounts of positive and negative strand RNAs were mixed in annealing buffer (100 mM potassium acetate, 4 mM MgCl₂ and 60 mM HEPES-KOH, pH 7.4), and incubated overnight at 37° C. to produce dsSKP1 or dsbZIP60 RNAs.

Probes for northern blots were prepared by adding 100 ng of NbSKP1, NtbZIP60 or PVX CP PCR fragments to a random priming reaction (North-2-South Biotin Random Prime Labeling kit, Pierce Biotechnology). Blots were incubated overnight with each probe at 55° C. and developed using Kodak Biolight film.

### REFERENCES

Aparicio F, Thomas C L, Lederer C, Niu Y, Wang D, Maule A J (2005) Virus induction of heat shock protein 70 reflects a general response to protein accumulation in the plant cytosol. Plant Physiol 138: 529-536
Bamunusinghe D, Hemenway C L, Nelson R S, Sanderfoot A A, Ye C M, Silva M A, Payton M, Verchot-Lubicz J (2009) Analysis of potato virus Xreplicase and TGBp3 subcellular locations. Virology 393: 272-285
Baumberger N, Tsai C H, Lie M, Havecker E, Baulcombe D C (2007) The Polerovirus silencing suppressor P0 targets ARGONAUTE proteins for degradation. Curr Biol 17: 1609-1614
Bhattarai K K, Li Q, Liu Y, Dinesh-Kumar S P, Kaloshian I (2007) The MI-1-mediated pest resistance requires Hsp90 and Sgt1. Plant Physiol 144:312-323
Bour S, Schubert U, Strebel K (1995) The human immunodeficiency virus type 1 Vpu protein specifically binds to the cytoplasmic domain of CD4:implications for the mechanism of degradation. J Virol 69: 1510-1520
Cardozo T, Pagano M (2004) The SCF ubiquitin ligase: insights into a molecular machine. Nat Rev Mol Cell Biol 5: 739-751
Chan S W, Egan P A (2005) Hepatitis C virus envelope proteins regulate CHOP via induction of the unfolded protein response. FASEB J 19:1510-1512
Chiu M H, Chen I H, Baulcombe D C, Tsai C H (2010) The silencing suppressor P25 of Potato virus X interacts with Argonaute 1 and mediates its degradation through the proteasome pathway. Mol Plant Pathol 11: 641-649
Costa M D, Reis P A, Valente M A, Irsigler A S, Carvalho C M, Loureiro M E, Aragao F J, Boston R S, Fietto L G, Fontes E P (2008) A new branch of endoplasmic reticulum stress signaling and the osmotic signal converge on plant-specific asparagine-rich proteins to promote cell death. J Biol Chem 283: 20209-20219
Denecke J, Carlsson L E, Vidal S, Ho\ddot{}glund AS, Ek B, van Zeijl M J, Sinjorgo K M, Palva E T (1995) The tobacco homolog of mammalian calreticulin is present in protein complexes in vivo. Plant Cell 7: 391-406
Denecke J, Goldman M H, Demolder J, Seurinck J, Botterman J (1991) The tobacco luminal binding protein is encoded by a multigene family. Plant Cell 3: 1025-1035
Dong Y, Burch-Smith T M, Liu Y, Mamillapalli P, Dinesh-Kumar S P (2007) A ligation-independent cloning tobacco rattle virus vector for high throughput virus-induced gene silencing identifies roles for NbMADS4-1 and -2 in floral development. Plant Physiol 145: 1161-1170
Draghici H K, Varrelmann M (2009) Evidence that the linker between the methyltransferase and helicase domains of potato virus X replicase is involved in homologous RNA recombination. J Virol 83: 7761-7769
Ellgaard L, Helenius A (2003) Quality control in the endoplasmic reticulum. Nat Rev Mol Cell Biol 4: 181-191
Garci´a-Marcos A, Pacheco R, Martia´n~z J, Gonza´lez-Jara P, Di´az-Rui´z JR, Tenllado F (2009) Transcriptional changes and oxidative stress associated with the synergistic interaction between Potato virus X and Potato virus Y and their relationship with symptom expression. Mol Plant Microbe Interact 22: 1431-1444
Iwata Y, Fedoroff N V, Koizumi N (2008) Arabidopsis bZIP60 is a proteolysisactivated transcription factor involved in the endoplasmic reticulum stress response. Plant Cell 20: 3107-3121
Iwata Y, Koizumi N (2005a) An Arabidopsis transcription factor, AtbZIP60, regulates the endoplasmic reticulum stress response in a manner unique to plants. Proc Natl Acad Sci USA 102: 5280-5285
Iwata Y, Koizumi N (2005b) Unfolded protein response followed by induction of cell death in cultured tobacco cells treated with tunicamycin. Planta 220: 804-807
Iwata Y, Yoneda M, Yanagawa Y, Koizumi N (2009) Characteristics of the nuclear form of the Arabidopsis transcription factor AtbZIP60 during the endoplasmic reticulum stress response. Biosci Biotechnol Biochem 73: 865-869
Jefferson R A, Kavanagh T A, Bevan M W (1987) GUS fusions: beta-glucuronidase as a sensitive and versatile gene fusion marker in higher plants. EMBO J. 6: 3901-3907
Jelitto-Van Dooren EPWM, Vidal S, Denecke J (1999) Anticipating endoplasmic reticulum stress: a novel early response before pathogenesisrelated gene induction. Plant Cell 11: 1935-1944
Jin H, Li S, Villegas A Jr (2006) Down-regulation of the 26S proteasome subunit RPN9 inhibits viral systemic transport and alters plant vascular development. Plant Physiol 142: 651-661
Ju H J, Brown J E, Ye C M, Verchot-Lubicz J (2007) Mutations in the central domain of potato virus X TGBp2 eliminate granular vesicles and virus cell-to-cell trafficking. J Virol 81: 1899-1911
Ju H J, Samuels T D, Wang Y S, Blancaflor E, Payton M, Mitra R, Krishnamurthy K, Nelson R S, Verchot-Lubicz J (2005) The potato virus X TGBp2 movement protein associates with endoplasmic reticulumderived vesicles during virus infection. Plant Physiol 138: 1877-1895
Ju H J, Ye C M, Verchot-Lubicz J (2008) Mutational analysis of PVX TGBp3 links subcellular accumulation and protein turnover. Virology 375: 103-117
Krishnamurthy K, Heppler M, Mitra R, Blancaflor E, Payton M, Nelson R S, Verchot-Lubicz J (2003) The Potato virus X TGBp3 protein associates with the ER network for virus cell-to-cell movement. Virology 309: 135-151
Leborgne-Castel N, Jelitto-Van Dooren E P, Crofts A J, Denecke J (1999) Overexpression of BiP in tobacco alleviates endoplasmic reticulum stress. Plant Cell 11: 459-470
Lee L Y, Fang M J, Kuang L Y, Gelvin S B (2008) Vectors for multi-color bimolecular fluorescence complementation to investigate protein-protein interactions in living plant cells. Plant Methods 4: 24
Liu Y, Burch-Smith T, Schiff M, Feng S, Dinesh-Kumar S P (2004) Molecular chaperone Hsp90 associates with resistance protein N and its signaling proteins SGT1 and Rar1 to modulate an innate immune response in plants. J Biol Chem 279: 2101-2108
Liu Y, Schiff M, Serino G, Deng X W, Dinesh-Kumar S P (2002) Role of SCF ubiquitin-ligase and the COP9 signalosome in the N gene-mediated resistance response to Tobacco mosaic virus. Plant Cell 14: 1483-1496
Lu D P, Christopher D A (2008) Endoplasmic reticulum stress activates the expression of a sub-group of protein disulfide isomerase genes and AtbZIP60 modulates the response in Arabidopsis thaliana. Mol Genet Genomics 280: 199-210
Mahalingam R, Jambunathan N, Gunjan S K, Faustin E, Weng H, Ayoubi P (2006) Analysis of oxidative signalling induced by ozone in Arabidopsis thaliana. Plant Cell Environ 29: 1357-1371
Malim M H, Emerman M (2008) HIV-1 accessory proteins: ensuring viral survival in a hostile environment. Cell Host Microbe 3: 388-398
Medigeshi G R, Lancaster A M, Hirsch A J, Briese T, Lipkin W I, Defilippis V, Fru\ddot{}h K, Mason P W, Nikolich-Zugich J, Nelson J A (2007) West Nile virus infection activates the unfolded protein response, leading to CHOP induction and apoptosis. J Virol 81: 10849-10860
Mekuria T, Bamunusinghe D, Payton M, Verchot-Lubicz J (2008) Phloem unloading of potato virus X movement proteins is regulated by virus and host factors. Mol Plant Microbe Interact 21: 1106-1117
Mitra R, Krishnamurthy K, Blancaflor E, Payton M, Nelson R S, Verchot-Lubicz J (2003) The potato virus X TGBp2 protein association with the endoplasmic reticulum plays a role in but is not sufficient for viral cell-to-cell movement. Virology 312: 35-48
Murai-Takebe R, Noguchi T, Ogura T, Mikami T, Yanagi K, Inagaki K, Ohnishi H, Matozaki T, Kasuga M (2004) Ubiquitination-mediated regulation of biosynthesis of the adhesion receptor SHPS-1 in response to endoplasmic reticulum stress. J Biol Chem 279: 11616-11625
Navazio L, Mariani P, Sanders D (2001) Mobilization of Ca2+ by cyclic ADP-ribose from the endoplasmic reticulum of cauliflower florets. Plant Physiol 125: 2129-2138 Nomaguchi M, Fujita M, Adachi A (2008) Role of HIV-1 Vpu protein for virus spread and pathogenesis. Microbes Infect 10: 960-967
Oh D H, Kwon C S, Sano H, Chung W I, Koizumi N (2003) Conservation between animals and plants of the cis-acting element involved in the unfolded protein response. Biochem Biophys Res Commun 301: 225-230
Petroski M D, Deshaies R J (2005) Function and regulation of cullin-RING ubiquitin ligases. Nat Rev Mol Cell Biol 6: 9-20
Preston A M, Gurisik E, Bartley C, Laybutt D R, Biden T J (2009) Reduced endoplasmic reticulum (ER)-to-Golgi protein trafficking contributes to ER stress in lipotoxic mouse beta cells by promoting protein overload. Diabetologia 52: 2369-2373
Qi Y, Zhong X, Itaya A, Ding B (2004) Dissecting RNA silencing in protoplasts uncovers novel effects of viral suppressors on the silencing pathway at the cellular level. Nucleic Acids Res 32: e179
Ratcliff F, Martin-Hernandez A M, Baulcombe D C (2001) Technical Advance. Tobacco rattle virus as a vector for analysis of gene function by silencing. Plant J 25: 237-245
Sambrook J, Fritsch E F, Maniatis T (1989) Molecular Cloning: A Laboratory Manual, Ed 2. Cold Spring Harbor Laboratory Press, Cold Spring Harbor, N.Y.
Samuels T D, Ye C-M, Ju H-J, Motes C M, Blancaflor E B, Verchot-Lubicz J (2007) Potato virus X TGBp1 protein accumulates independently of TGBp2 and TGBp3 to promote virus cell-to-cell movement. Virology 367: 375-389
Schepetilnikov M V, Manske U, Solovyev A G, Zamyatnin A A Jr, Schiemann J, Morozov S Y (2005) The hydrophobic segment of Potato virus X TGBp3 is a major determinant of the protein intracellular trafficking. J Gen Virol 86: 2379-2391
Schott A, Ravaud S, Keller S, Radzimanowski J, Viotti C, Hilimer S, Sinning I, Strahl S (2010) Arabidopsis stromal-derived factor2 (SDF2) is a crucial target of the unfolded protein response in the endoplasmic reticulum. J Biol Chem 285: 18113-18121
Schubert U, Anto´n LC, Baci´k I, Cox J H, Bour S, Bennink J R, Orlowski M, Strebel K, Yewdell J W (1998) CD4 glycoprotein degradation induced by human immunodeficiency virus type 1 Vpu protein requires the function of proteasomes and the ubiquitin-conjugating pathway. J Virol 72: 2280-2288
Seo P J, Kim S G, Park C M (2008) Membrane-bound transcription factors in plants. Trends Plant Sci 13: 550-556
Shadwick F S, Doran P M (2007) Propagation of plant viruses in hairy root cultures: a potential method for in vitro production of epitope vaccines and foreign proteins. Biotechnol Bioeng 96: 570-583
Silva A T, Nguyen A, Ye C, Verchot J, Moon J H (2010) Conjugated polymer nanoparticles for effective siRNA delivery to tobacco BY-2 protoplasts. BMC Plant Biol 10: 291
Slepak T I, Tang M, Slepak V Z, Lai K (2007) Involvement of endoplasmic reticulum stress in a novel classic galactosemia model. Mol GenetMetab 92: 78-87
Surjit M, Jameel S, Lal S K (2007) Cytoplasmic localization of the ORF2 protein of hepatitis E virus is dependent on its ability to undergo retrotranslocation from the endoplasmic reticulum. J Virol 81: 3339-3345
Tardif K D, Mori K, Kaufman R J, Siddiqui A (2004) Hepatitis C virus suppresses the IRE1-XBP1 pathway of the unfolded protein response. J Biol Chem 279: 17158-17164
Urade R (2007) Cellular response to unfolded proteins in the endoplasmic reticulum of plants. FEBS J 274: 1152-1171
Urade R (2009) The endoplasmic reticulum stress signaling pathways in plants. Biofactors 35: 326-331
Urano F, Wang X, Bertolotti A, Zhang Y, Chung P, Harding H P, Ron D (2000) Coupling of stress in the ER to activation of JNK protein kinases by transmembrane protein kinase IRE1. Science 287: 664-666

Verchot-Lubicz J, Torrance L, Solovyev A G, Morozov S Y, Jackson A O, Gilmer D (2010) Varied movement strategies employed by triple gene block-encoding viruses. Mol Plant Microbe Interact 23: 1231-1247
Wang X H, Aliyari R, Li W X, Li H W, Kim K, Carthew R, Atkinson P, Ding S W (2006) RNA interference directs innate immunity against viruses in adult Drosophila. Science 312: 452-454
Whitham S A, Quan S, Chang H S, Cooper B, Estes B, Zhu T, Wang X, Hou Y M (2003) Diverse RNA viruses elicit the expression of common sets of genes in susceptible Arabidopsis thaliana plants. Plant J 33: 271-283
Williams B L, Lipkin W I (2006) Endoplasmic reticulum stress and neurodegeneration in rats neonatally infected with borna disease virus. J Virol 80: 8613-8626
Xu C, Bailly-Maitre B, Reed J C (2005) Endoplasmic reticulum stress: cell life and death decisions. J Clin Invest 115: 2656-2664
Yu C Y, Hsu Y W, Liao C L, Lin Y L (2006) Flavivirus infection activates the XBP1 pathway of the unfolded protein response to cope with endoplasmic reticulum stress. J Virol 80: 11868-11880
Zamyatnin A A Jr, Solovyev A G, Bozhkov P V, Valkonen J P, Morozov S Y, Savenkov E I (2006) Assessment of the integral membrane protein topology in living cells. Plant J 46: 145-154
Zamyatnin A A Jr, Solovyev A G, Sablina A A, Agranovsky A A, Katul L, Vetten H J, Schiemann J, Hinkkanen A E, Lehto K, Morozov S Y (2002) Dual-colour imaging of membrane protein targeting directed by poa semilatent virus movement protein TGBp3 in plant and mammalian cells. J Gen Virol 83: 651-662
Zhang K, Kaufman R J (2006) The unfolded protein response: a stress signaling pathway critical for health and disease. Neurology (Suppl 1) 66: S102-S109 Plant Viral Movement Protein Elicits the UPR Plant Physiol. Vol. 156, 2011 755

The foregoing Examples are intended to illustrate certain embodiments of the present disclosure but should not be interpreted as limiting in any way.

All referenced articles, patents and patent applications cited herein are hereby incorporated by reference in entirety.

The present disclosure is well adapted to carry out the objectives and attain the ends and advantages mentioned above as well as those inherent therein. While presently preferred embodiments have been described for purposes of this disclosure, numerous changes and modifications will be apparent to those of ordinary skill in the art. Such changes and modifications are encompassed within the spirit of the present disclosure as defined by the claims.

What is claimed is:

1. A method of treating, preventing or slowing viral infection in a plant, comprising
inhibiting expression of a bZIP60 gene in said plant.

2. The method of claim 1, wherein said step of inhibiting is performed by providing to said plant at least one agent that inhibits bZIP60 expression.

3. The method of claim 2, wherein said at least one agent that inhibits bZIP60 expression is selected from the group consisting of: Co-suppressive RNA, dsRNA, antisense RNA, hairpin RNA, intron containing hairpin RNA, an amplicon mediated interference agent, catalytic RNA, small inhibitory RNA and microRNA.

4. The method of claim 3, wherein said agent that inhibits bZIP60 expression is dsRNA.

5. The method of claim 1, wherein said viral infection is due to a positive-sense ssRNA virus.

6. The method of claim 5, wherein said positive-sense ssRNA virus is a member of the Flexiviridae family.

7. A method of treating, preventing or slowing viral infection in a plant, comprising
inhibiting activity of a bZIP60 protein in plant cells of said plant.

8. A transgenic plant that is genetically engineered to have impaired bZIP60 activity.

9. The transgenic plant of claim 7, wherein said plant is genetically engineered to lack a bZIP60 gene.

10. The transgenic plant of claim 8, wherein said plant is genetically engineered to contain and express
i) a mutant bZIP60 gene that is expressed at a level that is lower than a comparable wild type bZIP60 gene; or
ii) a mutant bZIP60 gene that expresses a bZIP60 protein that has a level of activity that is lower than that of a comparable wild-type bZIP60 protein.

11. The transgenic plant of claim 8, wherein said plant is genetically engineered to contain and express an at least one agent that inhibits:
i) expression of a bZIP60 gene; or
ii) activity of a bZIP60 gene product.

12. A method of inducing the unfolded protein response (UPR) in a cell, comprising
providing to said cell in a quantity of TGBp3 sufficient to induce said UPR in said cell.

13. The method of claim 11, wherein said TGBp3 is provided to endoplasmic reticulum (ER) of said cell.

14. The method of claim 11, wherein said cell is a cancer cell and induction of UPR results in apoptosis of said cell.

15. The method of claim 11, wherein said cell is a damaged neural cell and induction of UPR triggers pro-survival pathways in said cell.

16. The method of claim 11, wherein said cell is in a mammal.


SEQUENCE LISTING (26 sequences)

SEQ ID NO: 1 - DNA, 47 bp, Artificial Sequence
  Synthetic oligonucleotide primer
gttgacggttaagtttcaccatcaccatcaccattgatactcgaaag

SEQ ID NO: 2 - DNA, 48 bp, Artificial Sequence
  Synthetic oligonucleotide primer
ctttcgagtatcaatggtgatggtgatggtgaaacttaaccgttcaac

SEQ ID NO: 3 - DNA, 55 bp, Artificial Sequence
  Synthetic oligonucleotide primer
ggggacaagtttgtacaaaaaacaggcttcggatccatggaagtaaatacatatc

SEQ ID NO: 4 - DNA, 69 bp, Artificial Sequence
  Synthetic oligonucleotide primer
ggggaccactttgtacaagaaagctgggtctcagtggtgatggtgatgatggaaacttaaccgttcaac

SEQ ID NO: 5 - DNA, 51 bp, Artificial Sequence
  Synthetic oligonucleotide primer
ggggacaagtttgtacaaaaaagcaggctgtatggatattctcatcagtag

SEQ ID NO: 6 - DNA, 49 bp, Artificial Sequence
  Synthetic oligonucleotide primer
ggggacaagtttgtacaaaaaagcaggctgtatgtcagcaccagctagc

SEQ ID NO: 7 - DNA, 50 bp, Artificial Sequence
  Synthetic oligonucleotide primer
ggggaccactttgtacaagaaagctgggtgctatggccctgcgcggacat

SEQ ID NO: 8 - DNA, 51 bp, Artificial Sequence
  Synthetic oligonucleotide primer
ggggaccactttgtacaagaaagctgggtgttatggtggtggtagagtgac

SEQ ID NO: 9 - DNA, 29 bp, Artificial Sequence
  Synthetic oligonucleotide primer
gcaagcttgatcatgagcggagaattaag

SEQ ID NO: 10 - DNA, 30 bp, Artificial Sequence
  Synthetic oligonucleotide primer
gcggtaccagatccggtgcagattatttgg

SEQ ID NO: 11 - DNA, 81 bp, Artificial Sequence
  Synthetic oligonucleotide primer
ggggacaagtttgtacaaaaaagcaggcttcggatccatggaacaaaaaatttctaagatctgtccgcgcagggccatagg

SEQ ID NO: 12 - DNA, 57 bp, Artificial Sequence
  Synthetic oligonucleotide primer
ggggaccactttgtacaagaaagctgggtactactaatgactgctatgattgttacc

SEQ ID NO: 13 - DNA, 82 bp, Artificial Sequence
  Synthetic oligonucleotide primer
ggggacaagtttgtacaaaaaagcaggcttgatgaacagaaacttatttctggaagaagatctggccaaggtgcgcgaggtt

SEQ ID NO: 14 - DNA, 51 bp, Artificial Sequence
  Synthetic oligonucleotide primer
ggggaccactttgtacaagaaagctgggtcttaaagaaagtttctgaggcg

SEQ ID NO: 15 - DNA, 47 bp, Artificial Sequence
  Synthetic oligonucleotide primer
ggggacaagtttgtacaaaaaagcaggctgtcggtcacgtggctgtc

SEQ ID NO: 16 - DNA, 47 bp, Artificial Sequence
  Synthetic oligonucleotide primer
ggggaccactttgtacaagaaagctgggtggaaccagaagaccgtgg

SEQ ID NO: 17 - DNA, 24 bp, Artificial Sequence
  Synthetic oligonucleotide primer
cctgctttggttcatgggcatcat

SEQ ID NO: 18 - DNA, 23 bp, Artificial Sequence
  Synthetic oligonucleotide primer
cacatcacaattcccaaataatg

SEQ ID NO: 19 - DNA, 24 bp, Artificial Sequence
  Synthetic oligonucleotide primer
tgacatgccagacagttgcagaca

SEQ ID NO: 20 - DNA, 18 bp, Artificial Sequence
  Synthetic oligonucleotide primer
ttcgatctcatcctggct

SEQ ID NO: 21 - DNA, 24 bp, Artificial Sequence
  Synthetic oligonucleotide primer
aaagaccagctcatccgtggagaa

SEQ ID NO: 22 - DNA, 24 bp, Artificial Sequence
  Synthetic oligonucleotide primer
tgtggtttcatgaatgccagcagc

SEQ ID NO: 23 - DNA, 1612 bp, Arabidopsis thaliana
gaccatggcggaggaatttggaagcatagatttactcggagatgaagatttcttcttcgatttcgatccttcaatcgtaattgattctcttccggcggaggattttcttcagtcttcaccggattcatggatcggagaaatcgagaatcaattgatgaacgatgagaatcatcaagaggagagttttgtggaattggatcagcaatcggtttcagatttcatagcggatctactcgttgattatccaactagcgattctggctccgttgatttggcggctgataaagttctaaccgtcgattctcccgccgccgctgatgattccgggaaggagaattcggatttggttgttgagaagaagtctaatgattctggtagcgagattcatgatgatgatgacgaagaaggagacgatgatgctgtggctaaaaaacgaagaaggtatatctaattttactcttgtttgaaaagttagggtttatggatggattttgtatgtttgtgttgattttggtttcattaagattgttaaaagtgtgttatgctttgagaacaacattgagaactaggagatacgtatgtgtaacagtgaagatttgagaaacgagtgtttaaattttgcttgcgtatcttgtgatatgttcatgttattatgtttatgtatgtgtgacgttgaagagttgattgattatgagtgatgttgatttttgtttcatttagattgctataagtgtgttattgttttgataacaacattgagaactaatcatttaagtaaatgactgtttaaattttggttgcctttaattattgttaatcttgtgttaatattcatgagctgattatgttcaaacataggtatgtgtattttggttaacttaagattgttaaagccttttatgctatgaaaagaaactgaggtttgataattgggatgtgattgtggttcaggagagtaagaaatagagatgcggcggttagatcgagagagaggaagaaggaatatgtacaagatttagagaagaagagtaagtatctcgaaagagaatgcttgagactaggacgtatgcttgagtgcttcgttgctgaaaaccagtctctacgttactgtttgcaaaagggtaatggcaataatactaccatgatgtcgaagcaggagtctgctgtgctcttgttggaatccctgctgttgggttccctgctttggcttctgggagtaaacttcatttgcctattcccttatatgtcccacacaaagtgttgcctcctacgtccagaaccagaaaagctggttctaaacgggctcgggagtagtagcaaaccgtcttataccggcgttagtcggagatgtaagggttcgaggcctaggatgaaataccaaatcttaacccttgcggcgtgacaacgccttttttaactgcttcttttgcgcattttgagttgtagatgagtgtcttttagttttctctctcttgttttgtatttcgctgttgaaagttttctgtctaatatcgataagttaacagtgaatgtgggtcttatggttatggatgatatctatctaataatgcttctgcctttaaaatgttgattttgaggcataacttcaggt

SEQ ID NO: 24 - protein, 295 aa, Arabidopsis thaliana
MAEEFGSIDLLGDEDFFFDFDPSIVIDSLPAEDFLQSSPDSWIGEIENQLMNDENHQEESFVELDQQSVSDFIADLLVDYPTSDSGSVDLAADKVLTVDSPAAADDSGKENSDLVVEKKSNDSGSEIHDDDDEEGDDDAVAKKRRRRVRNRDAAVRSRERKKEYVQDLEKKSKYLERECLRLGRMLECFVAENQSLRYCLQKGNGNNTTMMSKQESAVLLLESLLLGSLLWLLGVNFICLFPYMSHTKCCLLRPEPEKLVLNGLGSSSKPSYTGVSRRCKGSRPRMKYQILTLAA

SEQ ID NO: 25 - protein, 70 aa, Potato virus X
MEVNTYLNAIILVLVVTIIAVISTSLVRTEPCVIKITGESITVLACKLDAETIKAIADLKPLSVERLSFH

SEQ ID NO: 26 - DNA, 213 bp, Potato virus X
atggaagtaaatacatatctcaacgcaatcatacttgtgcttgtggtaacaatcatagcagtcattagtacttccttagtgaggactgaaccttgtgtcatcaagattactggggaatcaatcacagtgttggcttgcaaattagatgcagaaactataaaagccattgccgatctcaagccactctccgttgaacggttaagtttccattga